(19) United States
(12) Patent Application Publication
(10) Pub. No.: US 2024/0084324 A1
(43) Pub. Date: Mar. 14, 2024

(54) METHODS FOR PREPARATION OF SHELF-STABLE PLASMID DNA/POLYCATION PARTICLES WITH DEFINED SIZES FOR CELL TRANSFECTION

(71) Applicant: The Johns Hopkins University, Baltimore, MD (US)

(72) Inventors: Hai-Quan Mao, Baltimore, MD (US); Yizong Hu, Baltimore, MD (US); Yining Zhu, Baltimore, MD (US)

(21) Appl. No.: 18/261,944

(22) PCT Filed: Feb. 16, 2022
(86) PCT No.: PCT/US2022/016583

§ 371 Date: Jul. 18, 2023

Related U.S. Application Data
(60) Provisional application No. 63/149,981, filed on Feb. 16, 2021.

(51) Int. Cl. C12N 15/86; B01F 25/25; C12N 7/00

(52) U.S. Cl. CPC C12N 15/86; B01F 25/25; C12N 7/00; B01F 2215/0431; B01F 2215/045; C12N 2740/15022; C12N 2740/15043; C12N 2740/15052

(57) ABSTRACT

Disclosed are the optimal composition and size of DNA/polycation particles for efficient transfection of viral production cells in both adherent and suspension cultures. The size-dependent feature of DNA/polycation particle-mediated transfection for particles between 50 nm and 1000 nm also is disclosed. A new scalable method based on kinetic control of DNA/polycation nanoparticle assembly to prepare shelf-stable particles with defined sizes between 50 nm and 1000 nm also is disclosed. The presently disclosed DNA/polycation particles yield superior and reproducible transfection activity and shelf stability and can be used as an off-the-shelf product.

### STATEMENT OF GOVERNMENTAL INTEREST

This invention was made with government support under grant EB018358 awarded by the National Institutes of Health. The government has certain rights in the invention.

### BACKGROUND

Gene therapy has become an increasingly valuable modality for treating congenital and acquired conditions, and prophylactic and treatment vaccines, with the number of ongoing clinical trials topping nearly 1,000 globally in 2020, and several recently approved products. Many of these therapies include the use of vectorized viruses based on lentivirus (LVVs), Milone and O'Doherty, 2018, and adeno-associated virus (AAVs). Wang et al., 2019. One of the most common methods to produce LVVs is transient transfection of a HEK293 packaging cell line or derivative thereof with plasmid DNAs (pDNAs) encoding viral accessory proteins and a transfer plasmid that contains the vector backbone. Merten et al., 2016. Benchmark transfection vehicles include calcium phosphate, Pear et al., 1993, lipofectamine, Dalby et al., 2004, and poly(ethylenimine) (PEI). Boussif et al., 1995. In a typical transfection procedure using PEI (see, for example, FIG. 1A), pDNA mixture and PEI are separately dissolved in a serum-reduced medium. Following manual mixing, the suspension is typically incubated for between 0-60 min to allow for complete polyelectrolyte complex (PEC) coacervation, after which it is added to the cultures. The pDNA/PEI particles facilitate cell entry, Mislick and Baldeschwieler, 1996; Rejman et al., 2005, endosomal escape, Bus et al., 2018, and nuclear transport, Pollard et al., 1998, of pDNAs, resulting in transcription of the viral RNAs, as well as expression of packaging and envelope proteins. Successful co-transfection of all species of pDNAs is required to yield functional LVVs. Additionally, scalable and reproducible production methods are essential to ensure consistent quality of LVVs and safe, efficacious therapeutic outcomes. van der Loo et al., 2015. Such quality production is only possible when the transfection process is fully controlled to yield high degrees of efficiency and consistency.

The widely adopted method to manually prepare pDNA/PEI particles immediately before transfection, however, suffers from high batch-to-batch variation, negatively affecting the reliability and efficiency of viral vector production. As illustrated in FIG. 1A, inconsistencies readily occur due to several factors including: (1) complexity associated with assembly of particles composed of multiple pDNAs of different lengths; (2) difficulty in achieving uniform mixing throughout the mixing vessel (spatial heterogeneity); (3) difficulty in maintaining a consistent pDNA/PET ratio during the sequential addition processes (temporal heterogeneity); and (4) varied incubation times of particles formed throughout the production process. More importantly, such a manual preparation process is prone to operator-dependent variability and is challenging to scale up. For example, LVV production at pharmaceutical batch sizes of hundreds of liters requires liter-scale mixing of pDNA and PEI solutions, raising challenges of mass transfer in liquid handling. Therefore, it is critical to develop an engineering approach to produce shelf-stable pDNA/PEI particles in a highly scalable and consistent fashion to ensure high transfection efficiency with ease-of-use features.

A flash nanocomplexation (FNC) technique for scalable production of pDNA/PEI nanoparticles has been previously reported, Santos et al., 2016. Discrete sub-100 nm nanoparticles have been successfully generated in a lyophilized form for systemic delivery applications in vivo. Hu et al., 2019. These small nanoparticles, however, are sub-optimal for in vitro transfection in viral vector production cell lines (i.e., HEK293T or HEK293F cells), showing only a fraction of the peak transfection efficiency of the particles obtained by a standard manual mixing method. Size-dependent transfection efficiency for particles of sizes beyond 100 nm, however, has rarely been previously reported, Ogris et al., 1998; Zhang et al., 2019, and little mechanistic understanding exists. The poor insight into size-dependent transfection efficiency of pDNA/PEI particles reflects the lack of methods to control the size and stability of these particles in the range of 200 nm to 1000 nm. Conventional pipette mixing or dropwise addition without control of assembly kinetics results in particles with unpredictable sizes and a high degree of instability.

### SUMMARY

In some aspects, the presently disclosed subject matter provides a method for preparing a plurality of polycation/polyanion complex nanoparticles, the method comprising:
(a) flowing a first stream comprising one or more water-soluble polycationic polymers at a first variable flow rate and a second stream comprising one or more polyanionic polymers at a second variable flow rate into a first flash nanocomplexation (FNC) mixer to form a plurality of nanoparticles having a first particle size;
(b) flowing a third stream comprising the plurality of nanoparticles having a first particle size at a third variable flow rate and a fourth stream comprising an assembly buffer at a fourth variable flow rate into a second FNC mixer to form a plurality of assembled nanoparticles;
(c) incubating the plurality of assembled nanoparticles formed in step (b) for a period of time to form a plurality of assembled nanoparticles having a second particle size; and
(d) flowing a fifth stream comprising the plurality of assembled nanoparticles having a second particle size at a fifth variable flow rate and a sixth stream comprising a stabilization buffer at a sixth variable flow rate into a third FNC mixer to form a plurality of polycation/polyanion complex nanoparticles.

In some aspects, the one or more water-soluble polycationic polymers are selected from the group consisting of polyethylenimine (PEI), chitosan, PAMAM dendrimers, protamine, poly(arginine), poly(lysine), poly(beta-aminoesters), cationic peptides and derivatives thereof. In certain aspects, the one or more water-soluble polycationic polymers is polyethylenimine.

In some aspects, the one or more water-soluble polyanionic polymers are selected from the group consisting of poly(aspartic acid), poly(glutamic acid), negatively charged block copolymers, heparin sulfate, dextran sulfate, hyaluronic acid, alginate, tripolyphosphate (TPP), oligo(glutamic acid), a cytokine, a protein, a peptide, a growth factor, and one or more nucleic acids.

In some aspects, the one or more nucleic acids are selected from the group consisting of an antisense oligonucleotide, cDNA, genomic DNA, guide RNA, plasmid DNA, vector DNA, mRNA, miRNA, piRNA, shRNA, and siRNA. In certain aspects, the one or more nucleic acids comprise plasmid DNA (pDNA) or a mixture of different species of plasmid DNA. In certain aspects, the one or more nucleic acids comprise mRNA.

In particular aspects, the one or more nucleic acids comprise a mixture of one or more plasmid DNAs, wherein the one or more plasmid DNAs are selected from the group consisting of a transfer plasmid and plasmid DNAs encoding a gag protein, a pol protein, a rev protein, and an env protein.

In some aspects, the transfer plasmid encodes a lentiviral vector.

In certain aspects, the lentiviral vector comprises a modified left (5′) lentiviral LTR comprising a heterologous promoter, a Psi packaging sequence (105 +), a cPPT/FLAP, an RRE, a promoter operably linked to a polynucleotide encoding a therapeutic transgene, and a modified SIN (3′) lentiviral LTR.

In other aspects, the env protein comprises a VSV-g envelope glycoprotein.

In some aspects, the first variable flow rate, the second variable flow rate, the third variable flow rate, the fourth variable flow rate, the fifth variable flow rate, and the sixth variable flow rate are each independently between about 5 to about 400 mL/min.

In some aspects, the first particle size has a range between about 40 nm to about 120 nm. In certain aspects, the plurality of nanoparticles having a first particle size are formed under conditions at a pH of about 2.0 to 4.0, and a conductivity of about 0.05 to 2.0 mS cm⁻¹.

In certain aspects, the plurality of nanoparticles formed in step (b) are incubated at about room temperature (22±4° C.) for a period of time. In particular aspects, the period of time ranges from about 0.2 to about 5 hours.

In some aspects, the plurality of assembled nanoparticles having a second particle size are formed under conditions at a pH of about 6.0 to 8.0, and a conductivity of about 2.0 to 25.0 mS cm⁻¹. In certain aspects, the assembly buffer comprises phosphate buffered saline. In particular aspects, the phosphate buffered saline comprises one or more of NaCl, KCl, Na₂HPO₄, KH₂PO₄, and combinations thereof.

In certain aspects, the second particle size has a range between about 300 nm to about 500 nm.

In some aspects, the plurality of polycation/polyanion complex nanoparticles of step (d) are formed under conditions at a pH of about 2.0 to 4.0, and a conductivity of about 1.0 to 15.0 mS cm⁻¹. In certain aspects, the stabilization buffer comprises at least one sugar. In particular aspects, the sugar comprises trehalose. In yet more particular aspects, the one or more sugars comprise between about 10% to about 30% w/w of trehalose. In some aspects, the stabilization buffer comprises HCl.

In some aspects, the method further comprises lyophilizing or freezing the particles at about −80° C. for storage.

In other aspects, the presently disclosed subject matter provides a method for preparing a viral vector, the method comprising contacting one or more cells with a polycation/polyanion complex nanoparticle prepared by the presently disclosed methods or the presently disclosed plurality of polycation/polyanion complex nanoparticles. In some aspects, the method comprises dosing the plurality of polycation/polyanion complex nanoparticles to a monolayer culture of the one or more cells or a suspension culture of the one or more cells. In particular aspects, the one or more cells comprise HEK293 cells. In particular aspects, the one or more cells comprise HEK293S cells, HEK293T cells, HEK293F cells, HEK293FT cells, HEK293FTM cells, HEK293SG cells, HEK293SGGD cells, HEK293H cells, HEK293E cells, HEK293MSR cells, or HEK293A cells. In particular aspects, the one or more cells comprise HEK293T cells. In more particular aspects, the one or more cells comprise HEK293T cells adapted for suspension culture.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE FIGURES

The patent or application file contains at least one drawing executed in color. Copies of this patent or patent application publication with color drawing(s) will be provided by the Office upon request and payment of the necessary fee.

Having thus described the presently disclosed subject matter in general terms, reference will now be made to the accompanying Figures, which are not necessarily drawn to scale, and wherein:

FIG. 1A, FIG. 1B, FIG. 1C, and FIG. 1D demonstrates that the size of pDNA/PEI particles dominates the transfection efficiency. FIG. 1A is a schematic of preparation of pDNA/PEI particles and transfection process for production of LVVs. Each exclamation mark indicates a potential source for batch-to-batch variations in transfection outcomes, which can be addressed by engineering the properties and preparation processes of pDNA/PEI particles; FIG. 1B shows transfection efficiencies (characterized as transgene expression levels of the luciferase reporter) in a monolayer culture of HEK293T cells as a function of pDNA concentration at the mixing step and incubation time (0 to 60 min) before dosage. For the group of mixing at a DNA concentration of 5, 10 or 20 μg mL⁻¹, the particles were diluted 5, 10 or 20 times, respectively, to 1 μg mL⁻¹ to dose cells; FIG. 1C shows the change in the average size (z-average diameter given by dynamic light scattering, DLS) of pDNA/PEI particles following mixing of pDNA and PEI solutions in Opti-MEM. The growth kinetics is dependent on the concentration of pDNA. The error bars were derived from three independent experiments, demonstrating reproducibility and predictability under the experimental conditions used; FIG. 1D shows the direct correlation between transfection efficiency and the z-average particle size based on data points from all experiments (from FIG. 1B and FIG. 1C) with varying pDNA concentrations and incubation times;

FIG. 2A, FIG. 2B, FIG. 2C, FIG. 2D, FIG. 2E, FIG. 2F, and FIG. 2G show the process for production of size-controlled pDNA/PEI particles in the range of 60 nm to 1000 nm through control of assembly kinetics and surface charge modulation. FIG. 2A, is a schematic demonstration of the stepwise kinetic growth and quench. FIG. 2B, shows the predicable size growth induced under different concentrations of PBS. FIG. 2C, demonstrates that particle size growth was quenched by dilution with 20 mM HCl in 19% (w/w) trehalose solution at different time points along the growth curve in 1×PBS. FIG. 2D, is the z-average diameter distributions measured by DLS of a series of stabilized particles with distinct sizes. FIG. 2E, The zeta-potential, and bound PEI content (measured by N/P ratio) changed along with the growth and stabilization steps. The particles in the sham control were treated by premixed 1×PBS and 20 mM HCl solutions, and the size stayed unchanged (66 nm) after the treatment. FIG. 2F, TEM images of the particles obtained under the conditions of FIG. 2F-1. Original 66-nm nanoparticles as the building blocks; FIG. 2F-2. Stabilized particles with an average size of 120 nm; FIG. 2F-3 and FIG. 2F-4. Stabilized particles with an average size of 180 nm; FIG. 2F-5 and FIG. 2F-6. Stabilized 400-nm particles with an enlarged view to one of the particles; FIG. 2F-7. Another enlarged 400-nm particle with less salt precipitation (white speckles) in the negatively stained region. FIG. 2G, shows the effect of HCl concentration used in the quenching step on size stability, DNA protection, and transfection efficiency of the 400-nm particles. Note that the percentage axis is inverted to spread data points, showing that a high HCl concentration resulted in size shrinkage and loss of DNA. In FIG. 2B and FIG. 2C, the error bars were derived by three independent experiments, demonstrating predictability and reproducibility of the process. In FIG. 2G, the error bars were derived by three replicates within a single experiment;

FIG. 3A, FIG. 3B, and FIG. 3C show the transfection efficiencies of stable particles with controlled sizes ranging from 60 nm to 1000 nm. FIG. 3A, shows the efficiency of transgene expression of luciferase as a reporter. FIG. 3B and FIG. 3C show the efficiency of transgene expression of GFP is shown in FIG. 3B for percentage of GFP-positive cells and FIG. 3C for the mean fluorescent intensity in the population of GFP-positive cells. For monolayer culture of HEK293T cells, the cells were harvested and lysed at 24-h post-transfection, and the error bars present the standard deviation from 4 plate wells as replicates in a single experiment; For suspension culture of HEK293F cells, the cells were harvested and lysed at 48 h post-transfection, and the error bars present the standard deviation of 3 or 4 independent experiments (each was conducted in a single well of a 12-well plate);

FIG. 4A, FIG. 4B, FIG. 4C, FIG. 4D, FIG. 4E, FIG. 4F, FIG. 4G, FIG. 4H, FIG. 4I, FIG. 4J, FIG. 4K, and FIG. 4L show the quantitative Cellomics high-content analysis (HCA) of cellular uptake and endosomal escape by particles with different sizes. FIG. 4A shows the image analysis modality to analyze fixed cells directly in the tissue culture plates. Representative images are shown in FIG. 4B at 2 h and FIG. 4C 4 h after incubation with particles at different sizes. Quantitative results are presented in terms of FIG. 4D; particle spot characteristics (area and intensity) directly suggesting successful size control during particle-cell interactions; FIG. 4E, Gal8 spot characteristics (area and intensity) indicating formation of larger endosomal vesicles by larger particles; FIG. 4F, Frequency of detected particles and Gal8 spots in cells at 2 h; FIG. 4G, Average total particle intensity per cell at all time points as a representative measure of total particle uptake quantity; FIG. 4H, Average number of Gal8 spots per cell at all time points as an indication of endosomal escape level, serving as a predictive index for transfection efficiency according to previous reports using this assay; FIG. 4I, Proposed quantitative measure for overall endosomal escape degree, i.e., average total Gal8 spot intensity per cell, due to different Gal8 spot characteristics observed for different particle sizes; FIG. 4J, Transfection efficiencies (luciferase reporter expression level) as a result of incubation with particles at different sizes for different periods of time, which correlated well with the trends of total cellular uptake and endosomal escape levels; FIG. 4K, Regardless of the particle size, fitting the overall endosomal escape level (Y axis) of all plate well-averaged data points against the overall cellular uptake level (X axis) shows a strong positive correlation at 2 to 4 h post-dosage. In the figure, n=21 wells for the fitted line of 1 h and n=42 wells for the fitted line of 2 h and 4 h; FIG. 4L, Fitting the endosomal escape level in a single cell (Y axis) of all cells assessed in the same well of the group of 200 nm (n=5400 cells), 400 nm (n=4693 cells), and 900 nm (n=4336 cells), against the cellular uptake level in the same single cell (X axis), shows a strong positive correlation. The figure was generated by overlapping the FlowJo-generated pseudo-color heatmaps showing cell distribution density with an arbitrary correlation curve plotted. In FIG. 4A, FIG. 4B, and FIG. 4C, all figures share the same scale bar=50 μm as shown in the Cy5 panels;

FIG. 5A, FIG. 5B, FIG. 5C, and FIG. 5D demonstrate the scale-up production of pDNA/PEI particles with controlled sizes and validation of transfection efficiency for LVV production in bioreactors. FIG. 5A shows tunable particle size growth kinetics as a function of ionic strength of the particle growth medium (i.e., PBS concentration, 0.3×, 0.4×, 0.45×, and 0.5× of the full ionic strength). FIG. 5B is a schematic of the scale-up production process enabled by conducting the mixing steps in CU mixers at a flow rate of higher than 40 ml min⁻¹. FIG. 5C shows the stability of the 400-nm particles at ambient temperature. FIG. 5D show the stability of the 400-nm particles at different time points during storage at −80° C. Particle suspension samples were thawed at ambient temperature before testing;

FIG. 6A and FIG. 6B show the correlation of pDNA payload with size of pDNA/PEI particles. The theoretical pDNA payload (number of pDNA copies) per pDNA/PEI particle is shown when assuming the length of the pDNA is 4 kbp, 7 kbp, or 10 kbp, in the size range of FIG. 6A, 30 nm to 100 nm; or FIG. 6B, 100 nm to 700 nm;

FIGS. 7A, 7B, 7C, and 7D are transmission electron microscopy (TEM) images of particles at the original or stabilized grown sizes. FIG. 7A, the building blocks of 60-nm nanoparticles produced by flash nanocomplexation method, Santos, J. L. et al., 2016. In a size growth process, the particles were stabilized at FIG. 7B, double (120 nm) or FIG. 7C, triple (180 nm) of the original size to show the growth mechanism of association at interfaces upon contact of the 60-nm nanoparticles; FIG. 7D, populational features of stabilized 400-nm particles among different fields of the TEM observation;

FIG. 8A, FIG. 8B, FIG. 8C, and FIG. 8D demonstrate the effect of ionic strength and pH of the particle growth medium on growth kinetics and uniformity. For testing the non-buffering salt component of PBS, 60-nm nanoparticles were challenged with 1×PBS-equivalent (150 mM) or elevated (200 mM) concentrations of NaCl without pH being influenced. This resulted in different FIG. 8A, size growth rates; and FIG. 8B, polydispersity index (uniformity measure provided by dynamic light scattering) or FIG. 8C size distribution (direct uniformity illustration provided by dynamic light scattering) of particles stabilized by 20 mM HCl in 19% w/w trehalose at different sizes. For testing the buffering component of PBS, 60-nm nanoparticles were challenged with different concentrations of NaOH to mimic the pH shift. The mixing resulted in FIG. 8D, different final pH in the solution and FIG. 8E, different sizing behavior by the particles;

FIG. 9A, FIG. 9B, FIG. 9C, FIG. 9D, FIG. 9E, and FIG. 9F show the limited particle size change in transfection medium. For cell transfection experiments, the stabilized particles at a pDNA concentration of 25 μg mL⁻¹ were diluted to 1 μg mL⁻¹ by mixing with the transfection medium (FreeStyle 293). The size change of particles upon this dilution step was monitored by dynamic light scattering (DLS) and shown in FIG. 9A, for 60 nm or 400 nm particles within 20 min; and FIG. 9B, for 60 nm, 200 nm, 400 nm, or 800 nm particles within 4 h. Fixation by uranyl acetate, Ohi et al., 2004, with subsequent TEM observation also was used to monitor the change of particles in the diluted form in the transfection medium. Representative images are shown for 60 nm nanoparticles FIG. 9C, shortly (20 min) upon dilution or FIG. 9D, at 3 h upon dilution; and for 400 nm particles FIG. 9E, shortly (20 min) upon dilution or FIG. 9F, at 3 h upon dilution;

FIG. 10A, FIG. 10B, FIG. 10C, and FIG. 10D show representative Cellomics images of B16F10-Gal8-GFP cells incubated with Cy5-pDNA NPs for different durations. FIG. 10A, 1 h; FIG. 10B, 2 h; FIG. 10C, 4 h; FIG. 10D, 8 h. Negative control: cells treated by the transfection medium (Opti-MEM) without particles for the same periods of time, for which no particle signal nor endosomal escape events (Gal8-GFP puncta) was detected. All figures share the same scale bar=100 μm shown in the control group. These figures showed the trends seen by Cellomics quantitative analysis clearly.

FIG. 11A and FIG. 11B are confocal laser scanning microscopy images of B16F10-Gal8-GFP cells incubated with Cy5-pDNA particles for 4 h. FIG. 11A, the 3D view from a z-stack experiment scanning every 0.15 μm height of the cells; FIG. 11B, representative layer images sampled in the middle height of the cells (top panel), or at the cell base (for 200 or 400-nm groups)/the cell top (for 900-nm group). Color schemes: Hoechst 33342 (blue); GFP-Gal8 (green); Cy5-DNA (purple); colocalization of GFP and Cy5 (white);

FIG. 12A, FIG. 12B, FIG. 12C, FIG. 12D, FIG. 12E, and FIG. 12F show the complete data set of the particle cellular uptake assessed by Cellomics. FIG. 12A, average particle spot area; FIG. 12B, average particle spot intensity; FIG. 12C, average number of particles detected per cell; FIG. 12D, average total particle intensity per cell (the indicator of total uptake amount), and FIG. 12E, percentage of particle spot-positive cells assessed by Cellomics high-content analysis at 1, 2, 4, and 8 h post-dosage of Cy5-labeled DNA particles;

FIG. 13A demonstrated that this tritium labeling assay could accurately assess the absolute pDNA amount regardless of the particle size when particles were stabilized at different sizes and subjected to the same volume treatment of reporter lysis buffer and SOLVABLE solution. FIG. 13B showed the same trend and relative relationships among groups as the semi-quantitative uptake assessment of fluorescence (average total particle intensity per cell, FIG. 4G) by Cellomics high-content analysis. This verified the uptake behaviors of particles at different sizes;

FIG. 14A, FIG. 14B, FIG. 14C, FIG. 14D, and FIG. 14E show the complete data set of the particle-induced endosomal escape assessed by Cellomics. FIG. 14A, average Gal8 spot area; FIG. 14B, average Gal8 spot intensity; FIG. 14C, average number of Gal8 spots detected per cell; FIG. 14D, average total Gal8 spot intensity per cell (the indicator of total endosomal escape level); and FIG. 14E, Gal8 spot-positive cell percentage assessed by Cellomics high-content analysis at 1, 2, 4, and 8 h post-dosage of Cy5-labeled DNA particles;

FIG. 15 shows metabolism activities of the cells incubated with the NPs at different sizes;

FIG. 16A, FIG. 16B, and FIG. 16C show positive scaling of endosomal escape and cellular uptake on a single-cell level. The cell density heat map showing the relationship between total Gal8 spot intensity (endosomal escape level) and total particle spot intensity (cellular uptake level) on a single-cell level, for cells incubated with particles at 200 nm, 400 nm, or 900 nm for FIG. 16A, 2 h; and FIG. 16B, 4 h. Note that the three plots in FIG. 16A were used to generate FIG. 4L by overlaying with each other. Similarly, the three plots in FIG. 16B were used to generate FIG. 16C;

FIG. 17A and FIG. 17B show cellular uptake in suspension culture of HEK293F cells. FIG. 17A, confocal laser scanning microscopy observations of cellular uptake of Cy5-pDNA particles at 2 h upon dosage. Color schemes: Hoechst 33342 (blue); CellMask Green (green). Note that this dye stained the whole cell body instead of cellular membrane, presumably due to cell membrane permeation by the fixative 4% paraformaldehyde. However it clearly marked the cell body for assessing cellular uptake; Cy5-pDNA (purple). All images share the same scale bar as that in the control image; FIG. 17B, cellular uptake kinetics of ³H-pDNA particles assessed at 1, 2, 4 and 24 h post-dosage;

FIG. 18A, FIG. 18B, FIG. 18C, and FIG. 18D demonstrate that scaling the size growth process at a DNA concentration of 200 mg mL⁻¹. FIG. 18A, Predicable size growth induced under different concentrations of PBS; FIG. 18B, particle size growth was halted by dilution with 20 mM HCl in 19% (w/w) trehalose solution at different time points along the growth curve with 0.75×PBS; FIG. 18C, the z-average diameter distributions measured by DLS of a series of stable particles with distinct sizes; FIG. 18D, the efficiency of transgene expression of luciferase as a reporter. In these experiments, the starting DNA concentration (out of FNC preparation of the small nanoparticles) was 200 μg mL⁻¹. These small building blocks held a larger size, 80 nm to 100 nm, depending on the plasmid lengths. Upon solution challenge, the concentration was down to 100 mg mL⁻¹ and was finally at 50 mg mL⁻¹ upon particle stabilization;

FIG. 19A, FIG. 19B, FIG. 19C, FIG. 19D, FIG. 19E, and FIG. 19F show: (FIG. 19A) The scheme of the formulation process of plasmid DNA/PEI particles with a defined size; (FIG. 19B) The peristaltic and reservoir-based set-up with confined impinging jet (CIJ) connected; The z-average diameter and polydispersity index (PDI) of nanoparticles generated by (FIG. 19C) 500 mL/min; (FIG. 19D) 1000 mL/min; and (FIG. 19E) 2000 mL/min as assessed by dynamic scattering; (FIG. 19F) The particle size growth kinetics of growing particles generated by different flow rates as assessed by dynamic light scattering; and

FIG. 20A, FIG. 20B, FIG. 20C, FIG. 20D, and FIG. 20E show: (FIG. 20A) The z-average particle diameter and (FIG. 20B) the polydispersity index of stabilized plasmid DNA/PEI particles out of Step 3 assessed by dynamic light scattering; (FIG. 20C) The DNA concentration in suspension of stabilized plasmid DNA/PEI particles out of Step 3; (FIG. 20D) The transfection efficiency comparison between standard lab-scale preparation and 1000 mL/min, as assessed by measurement of relative light unit (RLU) of transgene expression of luciferase; (FIG. 20E) The comparison of particle size distribution between standard lab-scale preparation and 1000 mL/min assessed by dynamic light scattering.

### DETAILED DESCRIPTION

The presently disclosed subject matter now will be described more fully hereinafter with reference to the accompanying Figures, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed. many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

There are currently no methods known in the art to generate concentrated, stable, off-the-shelf DNA/PEI particles within the size range of about 200 nm to about 1000 nm for efficient transfection of cells, e.g., HEK293 cells, to produce viral vectors. The presently disclosed subject matter provides such a method and simplifies and streamlines the transfection process to make it operator-independent and to facilitate the scale-up production of viral vectors. The presently disclosed subject matter solves the poor reproducibility and inconsistent yield in production of viral vectors via a transient transfection process. As a result, the production quality and consistency of viral vectors can be improved. The presently disclosed methods and formulations potentially will find a wide range of applications in process engineering for production of viral vectors at various scales. Other potential uses include ex vivo transfection of cells for regenerative therapy and immunotherapy.

The presently disclosed subject matter, in some embodiments, discloses the optimal composition and size of DNA/polycation particles for efficient transfection of viral production cells in both adherent and suspension cultures. The size-dependent feature of DNA/polycation particle-mediated transfection for particles between 50 nm and 1000 nm also is disclosed. A new scalable method based on kinetic control of DNA/polycation nanoparticle assembly to prepare shelf-stable particles with defined sizes between 50 and 1000 nm also is disclosed. In particular embodiments, the presently disclosed subject matter provides an off-the-shelf particle formulation that is between about 400 nm to about 500 nm in size. The presently disclosed DNA/polycation particles yield superior and reproducible transfection activity and shelf stability and can be used as an off-the-shelf product.

As noted hereinabove, size-dependent transfection activity for particles having a size greater than 100 nm has been only occasionally reported previously, Ogris et al., 1998; Zhang et al., 2019. A systematic understanding of the particle-size dependence of the transfection process, however, has not been undertaken. Initial investigations conducted in developing the presently disclosed subject matter identified that particle size, particularly particles greater than 100 nm, is a key parameter affecting the transfection efficiency in certain cell lines (e.g., HEK293T or HEK293F cells). The presently disclosed subject matter provides the first direct correlation of the transfection efficiency of pDNA/PEI particles with an average particle size ranging from about 60 nm to about 1000 nm and demonstrates that particle size is the common determinant of the transfection activity for particles prepared under different conditions, with, in some embodiments, an optimal particle size between about 400 nm to about 500 nm in the conditions used in the cell cultures.

To date, no methods for controlling the size and stability of pDNA/PEI particles in the range of about 200 nm to about 1000 nm have been reported. The pipette mixing or dropwise addition results in particles with unpredictable sizes and a high degree of instability. In contrast, the presently disclosed subject matter provides a scalable method to produce pDNA/PEI particles at any desired size between about 60 nm to about 1000 nm by controlling the growth kinetics and kinetic stability. Using this particle series, a quantitative analysis was conducted, which revealed the key rate limiting step of cellular uptake controlling the intracellular trafficking and transfection activity. To further improve the translational potential, an off-the-shelf formulation of 400-nm pDNA/PEI particles was developed. These particles exhibited superior shelf stability at −80° C. with preserved physical properties and transfection activity. Supplied as a ready-to-use form, this particle formulation was validated in multiple scales of production of lentiviral vectors and demonstrated a consistent yield that is equivalent to the optimized complexes freshly prepared by the conventional manual preparation method.

### A. Method for Preparing Polycation/Polyanion Complex Nanoparticles

Accordingly, in some embodiments, the presently disclosed subject matter provides a method for preparing a plurality of polycation/polyanion complex nanoparticles, the method comprising:
(a) flowing a first stream comprising one or more water-soluble polycationic polymers at a first variable flow rate and a second stream comprising one or more polyanionic polymers at a second variable flow rate into a first flash nanocomplexation (FNC) mixer to form a plurality of nanoparticles having a first particle size;
(b) flowing a third stream comprising the plurality of nanoparticles having a first particle size at a third variable flow rate and a fourth stream comprising an assembly buffer at a fourth variable flow rate into a second FNC mixer to form a plurality of assembled nanoparticles;
(c) incubating the plurality of assembled nanoparticles formed in step (b) for a period of time to form a plurality of assembled nanoparticles having a second particle size; and
(d) flowing a fifth stream comprising the plurality of assembled nanoparticles having a second particle size at a fifth variable flow rate and a sixth stream comprising a stabilization buffer at a sixth variable flow rate into a third FNC mixer to form a plurality of polycation/polyanion complex nanoparticles.

In some embodiments, the one or more water-soluble polycationic polymers are selected from the group consisting of polyethylenimine (PEI), chitosan, PAMAM dendrimers, protamine, poly(arginine), poly(lysine), poly(beta-aminoesters), cationic peptides and derivatives thereof. In certain embodiments, the one or more water-soluble polycationic polymers is polyethylenimine.

In some embodiments, the one or more water-soluble polyanionic polymers is selected from the group consisting of poly(aspartic acid), poly(glutamic acid), negatively charged block copolymers, heparin sulfate, dextran sulfate, hyaluronic acid, alginate, tripolyphosphate (TPP), oligo(glutamic acid), a cytokine, a protein, a peptide, a growth factor, and one or more nucleic acids.

In some embodiments, the one or more nucleic acids are selected from the group consisting of an antisense oligonucleotide, cDNA, genomic DNA, guide RNA, plasmid DNA, vector DNA, mRNA, miRNA, piRNA, shRNA, and siRNA. In certain embodiments, the one or more nucleic acids comprise plasmid DNA (pDNA) or a mixture of different species of plasmid DNA. In certain embodiments, the one or more nucleic acids comprise mRNA.

In particular embodiments, a mixture of pDNAs encode a transfer plasmid comprising a packageable viral vector and one or more viral structural/accessory proteins necessary and sufficient to produce a viral vector.

In some embodiments, the first variable flow rate, the second variable flow rate, the third variable flow rate, the fourth variable flow rate, the fifth variable flow rate, and the sixth variable flow rate are each independently between about 5 to about 400 mL/min.

In some embodiments, the first particle size has a range between about 40 nm to about 120 nm, including about 40, 45, 50, 55, 60, 65, 70, 75, 80, 85, 90, 95, 100, 105, 110, 115, and 120 nm. In certain embodiments, the plurality of nanoparticles having a first particle size are formed under conditions at a pH of about 2.0 to 4.0 and a conductivity of about 0.05 to 2.0 mS cm⁻¹.

In certain embodiments, the plurality of nanoparticles formed in step (b) are incubated at about room temperature (22±4° C.) for a period of time. In particular embodiments, the period of time ranges from about 0.2 to about 5 hours.

In some embodiments, the plurality of assembled nanoparticles having a second particle size are formed under conditions at a pH of about 6.0 to 8.0, and a conductivity of about 2.0 to 25.0 mS cm⁻¹. In certain embodiments, the assembly buffer comprises phosphate buffered saline. In particular embodiments, the phosphate buffered saline comprises one or more of NaCl, KCl, Na₂HPO₄, KH₂PO₄, and combinations thereof.

In certain embodiments, the second particle size has a range between about 300 nm to about 500 nm, including about 300, 310, 320, 330, 340, 350, 360, 370, 380, 390, 400, 410, 420, 430, 440, 450, 460, 470, 480, 490, and 500 nm.

In some embodiments, the plurality of polycation/polyanion complex nanoparticles of step (d) are formed under conditions at a pH of about 2.0 to 4.0, and a conductivity of about 1.0 to 15.0 mS cm⁻¹. In certain embodiments, the stabilization buffer comprises at least one sugar. In particular embodiments, the sugar comprises trehalose. In yet more particular embodiments, the one or more sugars comprise between about 10% to about 30% w/w of trehalose. In some embodiments, the stabilization buffer comprises HCl.

In some embodiments, the method further comprises lyophilizing or freezing the particles at about −80° C. for storage.

### B. Method for Preparing a Viral Vector

In other embodiments, the presently disclosed subject matter provides a method for preparing a viral vector, the method comprising contacting one or more cells with a polycation/polyanion complex nanoparticle prepared by the presently disclosed methods or the presently disclosed plurality of polycation/polyanion complex nanoparticles. In some embodiments, the method comprises dosing the plurality of polycation/polyanion complex nanoparticles to a monolayer culture of the one or more cells or a suspension culture of the one or more cells.

In particular embodiments, one or more cells are transfected with a polycationic/nucleic acid nanoparticle, e.g., a pDNA/PEI complex, contemplated herein to generate viral vector.

Illustrative examples of cells suitable for transfection with the nanoparticles contemplated herein include, but are not limited to CHO cells, BHK cells, MDCK cells, C3H 10T1/2 cells, FLY cells, Psi-2 cells, BOSC 23 cells, PA317 cells, WEHI cells, COS cells, BSC 1 cells, BSC 40 cells, BMT 10 cells, VERO cells, W138 cells, MRCS cells, A549 cells, HT1080 cells, 293 cells, B-50 cells, 3T3 cells, NIH3T3 cells, HepG2 cells, Saos-2 cells, Huh7 cells, HeLa cells, W163 cells, 211 cells, 211A cells, or derivatives thereof.

In preferred embodiments, cells suitable for transfection with the nanoparticles contemplated herein comprise HEK293 cells or a derivative thereof. Derivatives of HEK293 cells suitable for use in particular embodiments contemplated herein include, without limitation, HEK293S cells, HEK293T cells, HEK293F cells, HEK293FT cells, HEK293FTM cells, HEK293SG cells, HEK293SGGD cells, HEK293H cells, HEK293E cells, HEK293MSR cells, and HEK293A cells.

In other particular preferred embodiments, the one or more cells comprise HEK293T cells adapted to suspension culture.

In some embodiments, the viral vector is a retroviral vector. Illustrative examples of retroviral vectors suitable for use in particular embodiments contemplated herein include but are not limited to vectors derived from Moloney murine leukemia virus (M-MuLV), Moloney murine sarcoma virus (MoMSV), Harvey murine sarcoma virus (HaMuSV), murine mammary tumor virus (MuMTV), gibbon ape leukemia virus (GaLV), feline leukemia virus (FLV), Spumavirus, Friend murine leukemia virus, Murine Stem Cell Virus (MSCV) and Rous Sarcoma Virus (RSV)) and lentivirus.

In preferred embodiments, the viral vector is a lentiviral vector. Illustrative examples of lentiviral vectors suitable for use in particular embodiments contemplated herein include but are not limited to vectors derived from HIV (human immunodeficiency virus; including HIV type 1, and HIV type 2); visna-maedi virus (VMV) virus; the caprine arthritis-encephalitis virus (CAEV); equine infectious anemia virus (EIAV); feline immunodeficiency virus (Hy); bovine immune deficiency virus (BIV); and simian immunodeficiency virus (SIV).

In more preferred embodiments, lentiviral vectors are derived from HIV-1 or HIV-2.

In particular embodiment, a transfer plasmid encodes a lentiviral vector that comprises a left (5′) lentiviral LTR, a Psi packaging sequence (Ψ+), a central polypurine tract/DNA flap (cPPT/FLAP), a rev response element (RRE), a promoter operably linked to a polynucleotide encoding a therapeutic transgene, and a right (3′) lentiviral LTR. Lentiviral vectors may optionally comprise post-transcriptional regulatory elements including, but not limited to, polyadenylation sequences, insulators, a woodchuck hepatitis virus posttranscriptional regulatory element (WPRE), a hepatitis B virus (HPRE), and the like.

In particular embodiment, a transfer plasmid a lentiviral vector that comprises a modified left (5′) lentiviral LTR comprising a heterologous promoter, a Psi packaging sequence (Ψ+), a central polypurine tract/DNA flap (cPPT/FLAP), a rev response element (RRE), a promoter operably linked to a polynucleotide encoding a therapeutic transgene, and a modified (3′) lentiviral LTR.

In particular embodiments, a transfer plasmid a lentiviral vector that comprises a modified 5′ LTR wherein the U3 region of the 5′ LTR is replaced with a heterologous promoter to drive transcription of the viral genome during production of viral particles. Examples of heterologous promoters which can be used include, for example, viral simian virus 40 (SV40) (e.g., early or late), cytomegalovirus (CMV) (e.g., immediate early), Moloney murine leukemia virus (MoMLV), Rous sarcoma virus (RSV), and herpes simplex virus (HSV) (thymidine kinase) promoters.

In particular embodiments, a transfer plasmid a lentiviral vector that comprises a modified self-inactivating (SIN) 3′ LTR that renders the viral vector replication defective. SIN vectors comprise one or more modifications of the U3 region in the 3′ LTR to prevent viral transcription beyond the first round of viral replication. This is because the right (3′) LTR U3 region is used as a template for the left (5′) LTR U3 region during viral replication and, thus, the viral transcript cannot be made without the U3 enhancer-promoter. In particular embodiments, the 3′ LTR is modified such that the U3 region is deleted and the R and/or U5 region is replaced, for example, with a heterologous or synthetic poly(A) sequence, one or more insulator elements, and/or an inducible promoter.

In particular embodiments, one or more pDNAs encode a transfer plasmid comprising a packageable viral vector genome and one or more of the viral structural/accessory proteins selected from the group consisting of: gag, pol, env, tat, rev, vif, vpr, vpu, vpx, and nef. In preferred embodiments, the viral structural/accessory proteins are selected from the group consisting of: gag, pol, env, tat, and rev. In more preferred embodiments, the viral structural/accessory proteins are selected from the group consisting of: gag, pol, env, and rev or gag, pol, and env.

Viral envelope proteins (env) determine the range of host cells which can ultimately be infected and transformed by recombinant retroviruses generated from the cell lines. In the case of lentiviruses, such as HIV-1, HIV-2, SIV, FIV and EIV, the env proteins include gp41 and gp120.

Illustrative examples of env genes which can be employed in the invention include, but are not limited to: MLV envelopes, 10A1 envelope, BAEV, FeLV-B, RD114, SSAV, Ebola, Sendai, FPV (Fowl plague virus), and influenza virus envelopes. Similarly, genes encoding envelopes from RNA viruses (e.g., RNA virus families of Picornaviridae, Calciviridae, Astroviridae, Togaviridae, Flaviviridae, Coronaviridae, Paramyxoviridae, Rhabdoviridae, Filoviridae, Orthomyxoviridae, Bunyaviridae, Arenaviridae, Reoviridae, Birnaviridae, Retroviridae) as well as from the DNA viruses (families of Hepadnaviridae, Circoviridae, Parvoviridae, Papovaviridae, Adenoviridae, Herpesviridae, Poxyiridae, and Iridoviridae) may be utilized. Representative examples include, FeLV, VEE, HFVW, WDSV, SFV, Rabies, ALV, BIV, BLV, EBV, CAEV, SNV, ChTLV, STLV, MPMV, SMRV, RAV, FuSV, MH2, AEV, AMV, CT10, and EIAV.

In other embodiments, env proteins suitable for use in particular embodiments include, but are not limited to any of the following viruses: Influenza A such as H1N1, H1N2, H3N2 and H5N1 (bird flu), Influenza B, Influenza C virus, Hepatitis A virus, Hepatitis B virus, Hepatitis C virus, Hepatitis D virus, Hepatitis E virus, Rotavirus, any virus of the Norwalk virus group, enteric adenoviruses, parvovirus, Dengue fever virus, Monkey pox, Mononegavirales, Lyssavirus such as rabies virus, Lagos bat virus, Mokola virus, Duvenhage virus, European bat virus 1 & 2 and Australian bat virus, Ephemerovirus, Vesiculovirus, Vesicular Stomatitis Virus (VSV), Herpesviruses such as Herpes simplex virus types 1 and 2, varicella zoster, cytomegalovirus, Epstein-Bar virus (EBV), human herpesviruses (HHV), human herpesvirus type 6 and 8, Human immunodeficiency virus (HIV), papilloma virus, murine gammaherpesvirus, Arenaviruses such as Argentine hemorrhagic fever virus, Bolivian hemorrhagic fever virus, Sabia-associated hemorrhagic fever virus, Venezuelan hemorrhagic fever virus, Lassa fever virus, Machupo virus, Lymphocytic choriomeningitis virus (LCMV), Bunyaviridiae such as Crimean-Congo hemorrhagic fever virus, Hantavirus, hemorrhagic fever with renal syndrome causing virus, Rift Valley fever virus, Filoviridae (filovirus) including Ebola hemorrhagic fever and Marburg hemorrhagic fever, Flaviviridae including Kaysanur Forest disease virus, Omsk hemorrhagic fever virus, Tick-borne encephalitis causing virus and Paramyxoviridae such as Hendra virus and Nipah virus, variola major and variola minor (smallpox), alphaviruses such as Venezuelan equine encephalitis virus, eastern equine encephalitis virus, western equine encephalitis virus, SARS-associated coronavirus (SARS-CoV), West Nile virus, any encephaliltis causing virus.

In preferred embodiments, the env gene encodes a VSV-G envelope glycoprotein.

In some preferred embodiments, pDNA/PEI complexes contemplated herein comprise a transfer plasmid encoding a lentiviral vector comprising a modified left (5′) lentiviral LTR comprising a heterologous promoter, a Psi packaging sequence (Ψ+), a cPPT/FLAP, an RRE, a promoter operably linked to a polynucleotide encoding a therapeutic transgene, and a modified SIN (3′) lentiviral LTR; a plasmid encoding a lentiviral gag/pol, a plasmid encoding rev, and a plasmid encoding an env gene, preferably a VSV-G envelope glycoprotein.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

The “subject” treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein. The term “subject” also refers to an organism, tissue, cell, or collection of cells from a subject.

In general, the “effective amount” of an active agent or drug delivery device refers to the amount necessary to elicit the desired biological response. As will be appreciated by those of ordinary skill in this art, the effective amount of an agent or device may vary depending on such factors as the desired biological endpoint, the agent to be delivered, the makeup of the pharmaceutical composition, the target tissue, and the like.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount, or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

### Methods for Preparation of Shelf-Stable Plasmid DNA/Polycation Particles with Defined Sizes for Cell Transfection

### 1.1 Overview

Polyelectrolyte complex (PEC) particles assembled from plasmid DNA (pDNA) and poly(ethylenimine) (PEI) have been widely used to produce lentiviral vectors (LVVs) for gene therapy. The current batch-mode preparation for pDNA/PEI particles suffers from limited reproducibility and stability particularly in large-scale manufacturing processes, leading to difficulty in controlling the transfection outcomes and LVV yield. The presently disclosed subject matter identified the size of pDNA/PEI particles as a key determinant for a high transfection efficiency with an optimal size of 400 nm to 500 nm, due to a cellular uptake-limited mechanism. A kinetics-based approach was developed to assemble size-controlled (400 nm) and shelf-stable particles using 60-nm nanoparticles as building blocks. The production scalability of this bottom-up engineering process also is demonstrated. The preservation of colloidal stability and transfection efficiency was validated against unstable particles generated using an industry standard protocol. This particle manufacturing method effectively streamlines the viral manufacturing process and improves the production quality and consistency.

To that end, the presently disclosed subject matter provides the first direct correlation of the transfection efficiency of pDNA/PEI particles within a wide size range of 60 nm to 1000 nm, observing an optimal size of 400 nm to 500 nm in both adherent and suspension cultures. More particularly, the presently disclosed subject matter provides a scalable method to produce pDNA/PEI particles at any size between 60 nm and 1000 nm by bottom-up assembly of 60-nm nanoparticles through controlling growth kinetics and colloidal stability. Using this particle series, a quantitative analysis was conducted, which revealed that the key rate limiting step is size-dependent cellular uptake in the intracellular delivery process. To further improve translational potential, an off-the-shelf formulation of the optimized 400-nm pDNA/PEI particles was engineered, demonstrating superior shelf stability at −80° C. with preservation of physical properties and transfection activity. These particles in ready-to-use forms could be produced across multiple therapeutically-relevant scales. Importantly, when validated in industrial production systems these particles generated superior therapeutic LVVs compared to those resulting from standard manual particle preparation methods.

### 1.2 Results and Discussion

### 1.2.1 Particle Size Dominates Transfection Efficiency

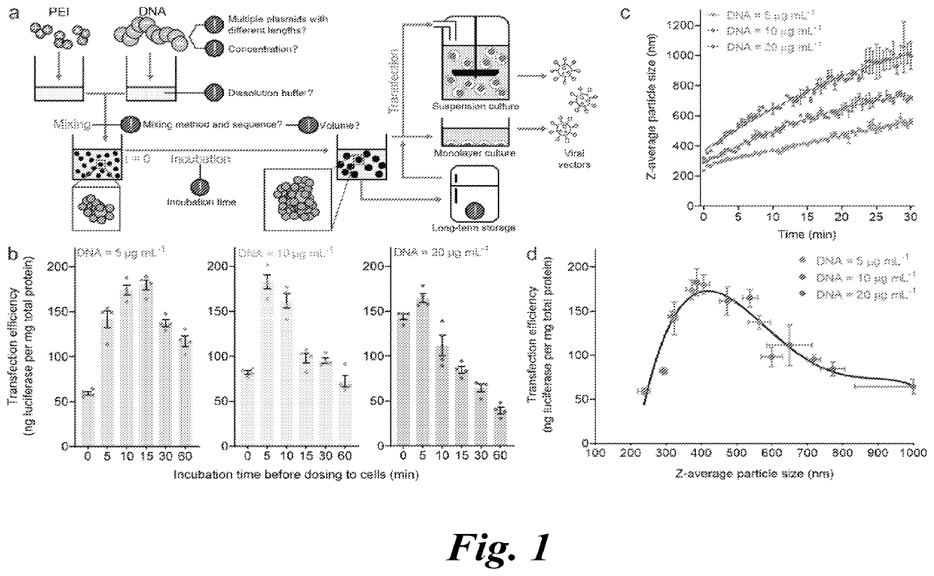

To demonstrate a typical transfection process for LVV production, where multiple species of pDNAs encoding different viral components are used, three pDNAs with a ratio of 10% (4.4 kb, non-coding), 45% (6.8 kb, gWiz-Luc luciferase reporter) and 45% (9.6 kb, non-coding) were dissolved in Opti-MEM medium. Following the scheme shown in FIG. 1A, a 10-second vortex was used as the mixing method after pipetting 100 μL of PEI solution (also in Opti-MEM) into 100 μL of pDNA solution (5, 10, or 20 μg mL⁻¹) at a nitrogen to phosphate (N/P) ratio of 5.5, followed by incubation at room temperature for 0 to 60 min before transfection tests in HEK293T cells. When the pDNA dose remained constant, i.e., 0.1 μg per 10⁴ cells at 1 μg mL⁻¹, the transfection efficiency as characterized by luciferase expression showed a bell-shaped relationship with incubation time, and the peak occurred at different incubation times for different DNA concentrations in particle preparation (FIG. 1B). With increasing DNA concentration, the time needed to achieve the highest transfection efficiency decreased. Next, the particle size was monitored as a function of incubation time upon mixing of pDNA and PEI solutions by dynamic light scattering (DLS) and found that the size increased in a predicable manner during incubation (FIG. 1C). When using incubation time as the bridge to reduce the number of variables, the transfection efficiency was plotted against the DLS-derived z-average particle diameter in FIG. 1D. A strong correlation emerged with a curve fitting all data points collected under different pDNA concentrations in particle preparation. This demonstrated the dominant dependence of transfection efficiency on the size of pDNA/PEI particles, and an optimal size range of 400 nm to 500 nm that would yield the highest transgene expression level.

1.2.2 Production of Stable pDNA/PEI Particles with Controlled Size in the Range of 60 to 1000 nm

The finding that particle size governs transfection efficiency motivated us to develop a method for producing shelf-stable pDNA/PEI particles with a controlled size of 400 nm to 500 nm. Experiments on transfecting cells using pDNA/PEI particles around this size range were reported previously, Ogris et al., 1998; Zhang et al., 2019; however, there is no specific effort reported to date on controlling particle size and uniformity in this range while maintaining stability.

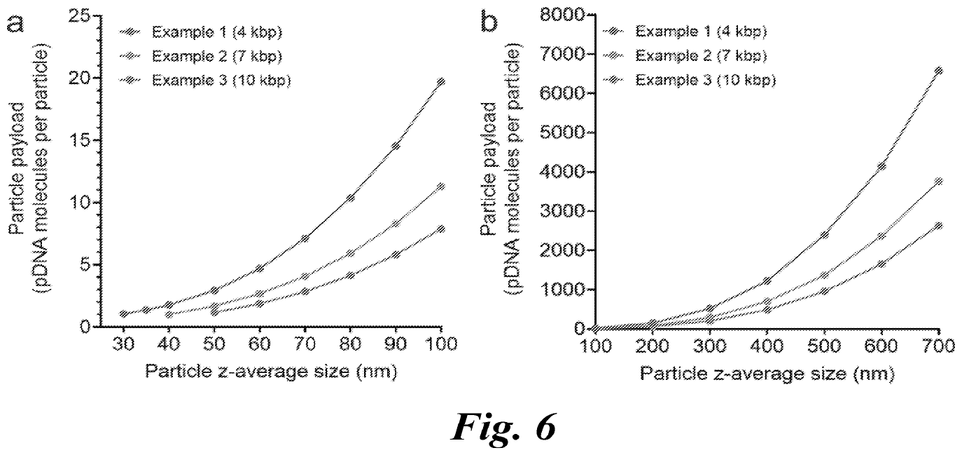

Controlling pDNA particle size in this range is particularly challenging. Previous characterization work using static light scattering, Hu et al., 2019, and using analytical ultra-centrifugation, Tockary et al., 2019, demonstrated that one pDNA/polycation nanoparticle that consists of a single pDNA molecule is only 20 nm to 50 nm in size, indicating that a 400- to 500-nm particle will need nearly thousands copies of pDNA to construct (FIG. 6).

During the particle assembly process, negatively charged pDNA collapses into a condensed state upon charge neutralization with positively charged PEI, Osada et al., 2010; the assembly kinetics is extremely fast with a time scale of 100s of milliseconds, whereas the diffusion rates of pDNA and the complexes are far slower. Thus, it is only possible to achieve a 400- to 500-nm size in a single step using an extremely high DNA concentration in the assembly process. It is estimated that the required concentration far exceeds 1 mg mL⁻¹, which would be exceedingly difficult to handle and scale up due to high viscosity. Hu et al., 2019.

It has been suggested previously, Hu et al., 2019, regarding the kinetics of the assembly of pDNA/PEI particles, that the weight-average molar mass of the particles is linearly proportional to the z-average particle diameter to the third power, regardless of the mixing condition. The results across different N/P ratios also were similar.

[M_{w},Da]=67.7×[D_{Z},nm]³+1.9×10⁶

The rough calculations adopt 650 Da per bp for double-stranded pDNA, a constant bound PEI fraction, Hu et al., 2019; Yue, Y. et al., 2011, which is equivalent to N/P=2.7 to 3.0, and a molecular weight of one repeat unit of linear PEI as 43 Da. Therefore, the total molar mass of a single pDNA with all its associated PEI can be determined to be around 880 Da per bp. The correlation of theoretical pDNA payload per pDNA/PEI particle and particle size can then be derived from the above equation. Considering common pDNAs bearing a functional expression cascade have a length range of 4 kbp to 10 kbp, three examples of pDNA with a length of 4 kbp, 7 kbp or 10 kbp are shown in FIG. 6A and FIG. 6B to demonstrate the payload-size correlation. Notably, a 400- to 500-nm particle contains roughly 1000 to 2500 pDNAs for 4 kbp pDNA, and 500 to 1000 pDNAs for 10 kbp pDNA, which are too high to assemble in a single step within the short particle assembly time.

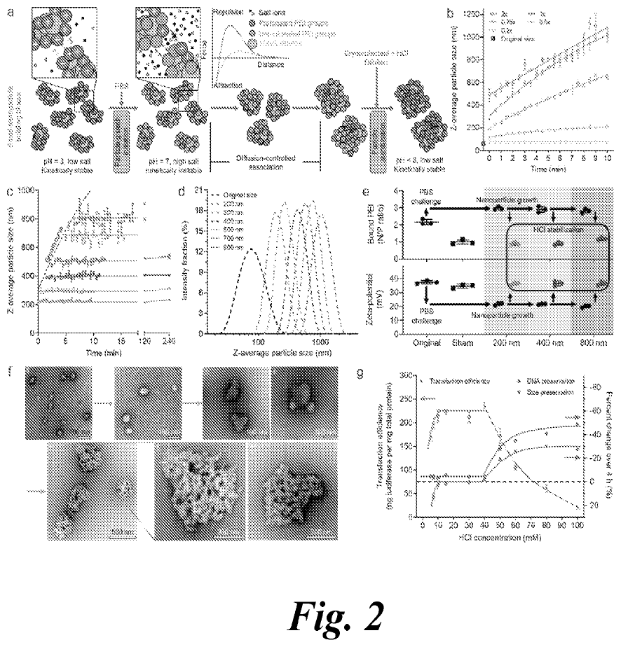

To circumvent this challenge, a bottom-up assembly strategy based on the characteristics of the kinetic growth of pDNA/PEI particles was developed (FIG. 2A). First, uniform small nanoparticles were prepared in a low-salt (conductivity approximately 0.4 mS cm⁻¹), low-pH (a pH of approximately 3) condition and these nanoparticles were used as the building blocks for secondary assembly. As per the buffering capacity of PEI, Curtis et al., 2016, over 80% of the secondary nitrogen groups are protonated (i.e., positively charged) at pH 3. The individual nanoparticles prepared under this condition are sufficiently stable against aggregation due to the net positive charges of the PEI molecules presenting on the particle surfaces.

When the medium is switched to pH 7, the nanoparticle surface becomes sufficiently deprotonated (zeta-potential drops from approximately +40 mV to +20 mV). Coupling the shortening of the Debye length associated with the residual surface charges from salt-induced charge screening, the medium condition change triggers particle association and size growth of nanoparticles. It is important to note that the ionic strength needs to be controlled at a level not to induce dissociation of the pDNA/PEI PECs, Bertschinger et al., 2006, rather only to initiate nanoparticle association. The particle growth is primarily driven by van der Waals force, with the rate determined by particle concentration and ionic strength of the medium. Particle growth is effectively quenched by reversing the pH to 3 (to re-protonate the particle surfaces) and by dilution to reduce the ionic strength, thus re-establishing the long-range Debye screening.

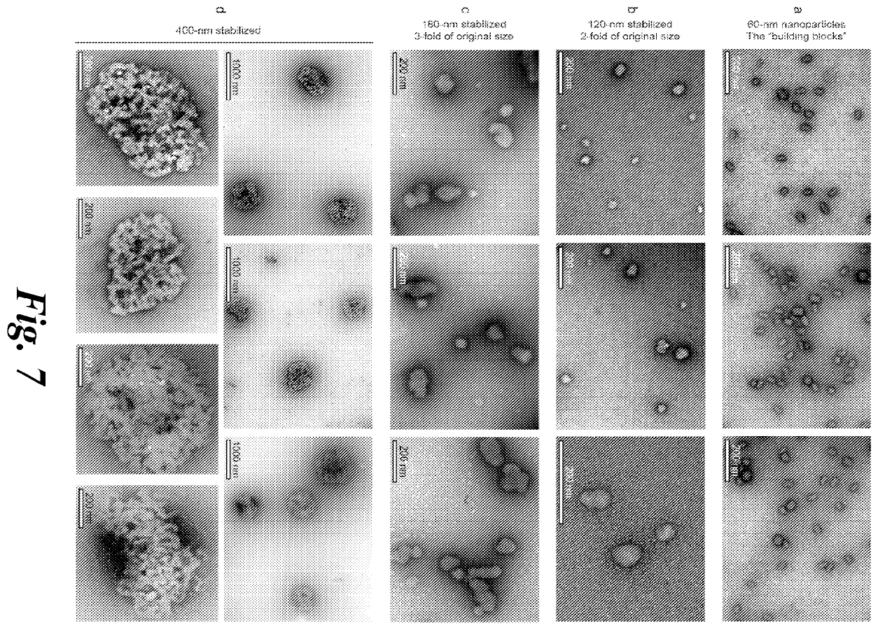

The building block pDNA/PEI nanoparticles were prepared using the FNC technique, Santos et al., 2016; Hu et al., 2019, in a confined impinging jet (CIJ) mixer, Johnson and Prud'homme, 2003; Hao et al., 2020, under high flow rate-induced turbulent mixing. Such a mixing condition reduces the characteristic mixing time for pDNA and PEI solutions to below the characteristic nanoparticle assembly time, to achieve uniform assembly kinetics and controlled nanoparticle size and composition. Hu et al., 2019. With an input pDNA concentration of 100 μg/mL, the pDNA/PEI nanoparticles held an average size of 66.0±1.0 nm measured by DLS and transmission electron microscopy (TEM) (FIG. 2B and FIG. 7A).

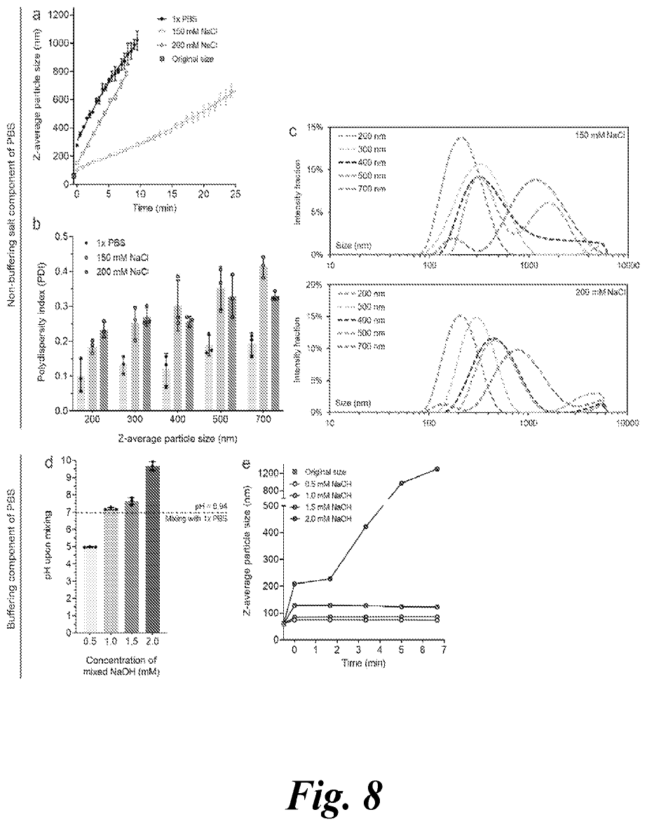

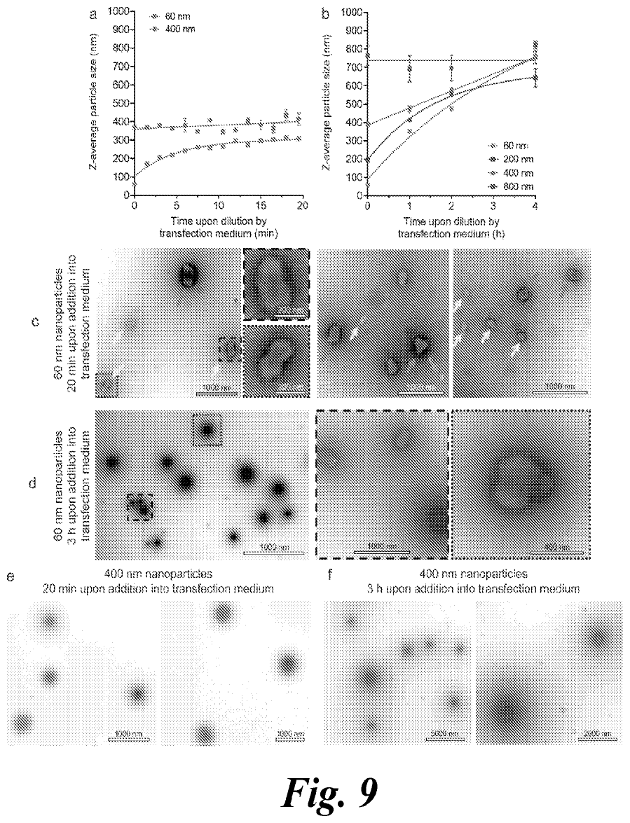

The proposed strategy was first tested in a small batch scale using pipetting as the particle assembly method. The nanoparticle suspension was challenged by mixing it with an equal volume of PBS, which initiated gradual size growth. The growth rate was dependent on the PBS concentration (FIG. 2B). It also was revealed that the buffering component of PBS was important to confer pH change and maintain particle uniformity; while the salt component primarily determined the growth kinetics (FIG. 8).

Proceeding with 1×PBS challenge and quenching the growth by mixing with an equal volume of 20 mM HCl in 19% w/w trehalose (a cryoprotectant) at different time points along its growth curve successfully stabilized the average particle size at 200, 300, 400, 500, 700 and 900 nm (FIG. 2C) with a high degree of uniformity (FIG. 2D). These sizes were intentionally selected as a proof of principle; any other desired sizes can be readily achieved by halting the growth at different time points. All particles with the average sizes of less than 500 nm were stable for at least 4 h at ambient temperature in the preparing medium (FIG. 2C). Larger particles continued growing, albeit at a much slower rate under these conditions, by about 100 nm in 4 h.

The composition of PBS could be categorized into two subsets: pH-buffering component (namely Na₂HPO₄ and KH₂PO₄) and non-buffering salt component (namely NaCl and KCl). When using only the non-buffering salt component of a 1×PBS at the same ionic strength to induce the growth of the 60-nm nanoparticles, it showed significantly slower growth rate comparing to that of 1×PBS (FIG. 8A). Using an elevated NaCl concentration of 200 mM raised the growth rate to a comparable level. NaCl alone, however, could not generate uniform particles. Upon stabilization of the particles by mixing with equal volume of 20 mM HCl in 19% w/w trehalose, the polydispersity index (PDI, FIG. 8B a higher uniformity correlates with a lower PDI value) and size distribution (FIG. 8C) demonstrated a heterogeneous nature of the particles from a challenge by both NaCl concentrations. This was in sharp contrast to particles prepared by induction with 1×PBS, where significantly lower PDIs (FIG. 8B) and distinct size peaks (FIG. 2D) were obtained. Pertaining to the size growth mechanism, insufficient deprotonation of PEI caused additional barriers for particle association. This may render the system controlled concurrently by diffusion, activation, and potentially steric effects that are a function of particle size.

Upon mixing the 60-nm nanoparticles (at a DNA concentration of 100 μg mL⁻¹) with equal volume of 1×PBS, the pH of the suspension increased from approximately 3 to 7. When pH was altered to 5 to 8 by directly mixing with NaOH solutions without involvement of non-buffering salt (FIG. 8D), no size growth was observed (FIG. 8E). The size of the particles did shift upwards, but this observation might be a result of shape transformation associated with gradual deprotonation of PEI in a low salt environment. Santos, J. L. et al., 2016. Only a pH shift above 9 induced a delayed, uncontrollably fast size growth. Given that the protonation fraction of PEI is still slightly higher than 40% in the pH range of 7-8, this data set indicated that partial deprotonation was not sufficient to induce particle association, and screening of the remaining charge on particle surface was vital. Thus, the non-buffering salt in PBS served as the major determinant of growing kinetics.

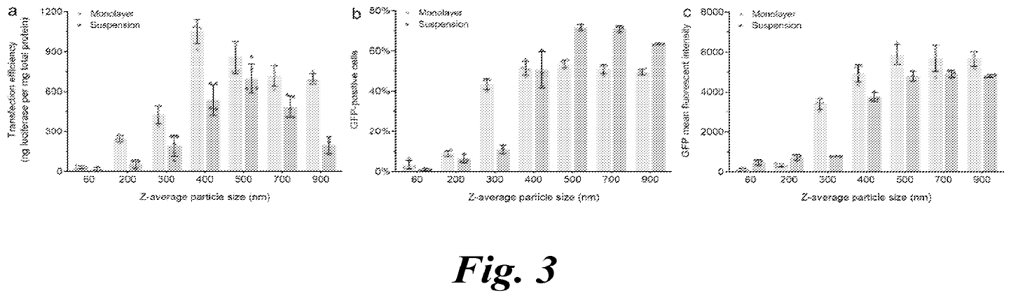

The proposed size control mechanism was verified by zeta-potential measurements through phase analysis light scattering (PALS) and PEI composition assessments, Bertschinger et al., 2004, of the growing and stabilized particles (FIG. 2F). Upon challenge by 1×PBS, the zeta-potential dropped from +37 mV to +20 mV and returned to the original level once stabilized by the HCl solution. The two steps of changes were associated with de-protonation of PEI that resulted in more PEI molecules involving in charge neutralization with pDNA (an increase in bound PEI), and re-protonation of PEI to reverse the effect. During the controlled growth step, a zeta-potential of +20 mV was sufficient to overcome the potential energy barrier in the presence of appropriate ionic strength. Transmission electron microscopy (TEM) analysis confirmed the DLS measurements showing the morphology and the nature of association of the original individual nanoparticles merged at their interfaces (FIG. 2F-2, FIG. 2F-3, FIG. 2F-4 and FIG. 7B, FIG. 7C). The stabilized 400-nm particles presented as uniformly distributed agglomerate constructs with a high level of uniformity (FIG. 2C-5, FIG. 2C-6, FIG. 2C-7, and FIG. 7D). An appropriate HCl concentration for the growth quenching is close to that required to fully protonate the buffering salts added during the growth phase. Lower or higher concentrations resulted in ineffective stabilization, or particle shrinkage and DNA degradation, respectively (FIG. 2G). These stability metrics also were confirmed by the transfection efficiencies of these particles.

1.2.3 Transfection Efficiencies of Stable pDNA/PEI Particles with Controlled Sizes

The stabilized particles were dosed to a monolayer culture of HEK293T cells or a suspension culture of HEK293F cells to test their transfection efficiency using pDNA either encoding luciferase or GFP as a reporter. For the transfection experiments, the particle suspension was diluted to a concentration of 1 μg pDNA mL⁻¹, which effectively limited further size growth under the transfection condition in a pH-neutral and high-salt medium (FIG. 9).

It is important to note that particles could grow to larger sizes upon addition into the transfection medium (physiological salt condition, neutral pH) when they are interacting with the cells. The growth kinetics is slow due to diffusion limitation imposed by the dilution (25 fold from 25 μg pDNA mL⁻¹ to 1 μg pDNA mL⁻¹), and the size growth was more profound with smaller initial size (FIG. 9A, FIG. 9B). Taking the 60 nm or 200 nm particles as examples, however, the significant size growth within the time scale of incubation with cells (4 h for monolayer transfection and the entire culture period, i.e., 48 h for suspension transfection) still did not make them more effective (FIG. 3). This observation prompted us to examine if the size growth in the diluted form in transfection medium was different than the size growth by the controllable method (FIG. 2). The TEM images confirms that: (1) There was indeed a size growth for 60 nm nanoparticles. Particles with a wide range of sizes could be observed shortly upon medium dilution, with green arrows pointing to nanoparticles below 100 nm, yellow arrows pointing to particles between 100 nm and 250 nm, and red arrows pointing to particles above 250 nm (FIG. 9C). At the same time point, DLS gave a z-average size reading of 250 nm. After 3 h, the particles grew to heterogeneously larger ones (FIG. 9D) without significant differences in morphology comparing with stabilized particles in the size range of 400 nm (FIG. 2F, and FIG. 7D); (2) The 400-nm particles clearly preserved the original morphology as sampled 20 min or 3 h upon dilution in transfection medium (FIG. 8E, 8F). There was also size growth along the incubation.

The TEM observations confirmed the DLS monitoring results that a slow size growth did occur in the transfection medium. The reasons for the ineffectiveness of particles below 300 nm in size before dosing to transfection medium, even though with the ability of size growth in the medium, however, remain elusive. This warrants future investigations and highlights the importance of controlling the particle size before transfection dosage in a stable manner.

The observation of pDNA/PEI particles was enabled by negative staining using uranyl acetate. Stabilized particles with a positively charged PEI surface repel the positively charged dye, giving sharp contrast in the images showing excellent 3D structures (FIG. 2F, FIG. 7). In the transfection medium, the surface charge is largely screened. Dye penetration and its interactions with pDNA were significant, rendering the particle core concentrated with the dye and too dark to observe. In such case, all images in FIG. 9 were applied a gamma correction with a power of 2 to 4 to make the darker areas lighter. This sacrificed the resolution and quality of the images obtained but did not affect the general discussions and conclusions.

The luciferase activity readouts (FIG. 3A) verified the optimal size of 400 nm in a monolayer culture as observed in FIG. 1D; and indicated an optimal size of 500 nm in a suspension culture. The GFP readouts (FIG. 3B, FIG. 3C) suggested that a qualitative efficiency jump occurred between 200 nm and 300 nm and plateaued at 400 nm in the monolayer culture, while it occurred from 300 nm to 400 nm and plateaued at 500 nm in the suspension culture. The trends from luciferase and GFP reporters agreed well for particle sizes from 60 nm to 500 nm but differed for particles over 500 nm, presumably due to different expression kinetics, protein stability, and assessment methods of the two reporters. Clearly, a small particle size <200 nm was ineffective in transfection even though they eventually grew in the transfection medium to a larger size (FIG. 8). In the suspension culture, the particles were not removed from the transfection medium, and had access to cells for the entire culture period of 48 h. Therefore, the results demonstrated the importance of controlling the particle size before dosing to the cells.

### 1.2.4 Intracellular Trafficking Mechanisms for Size-Dependent Transfection Efficiency

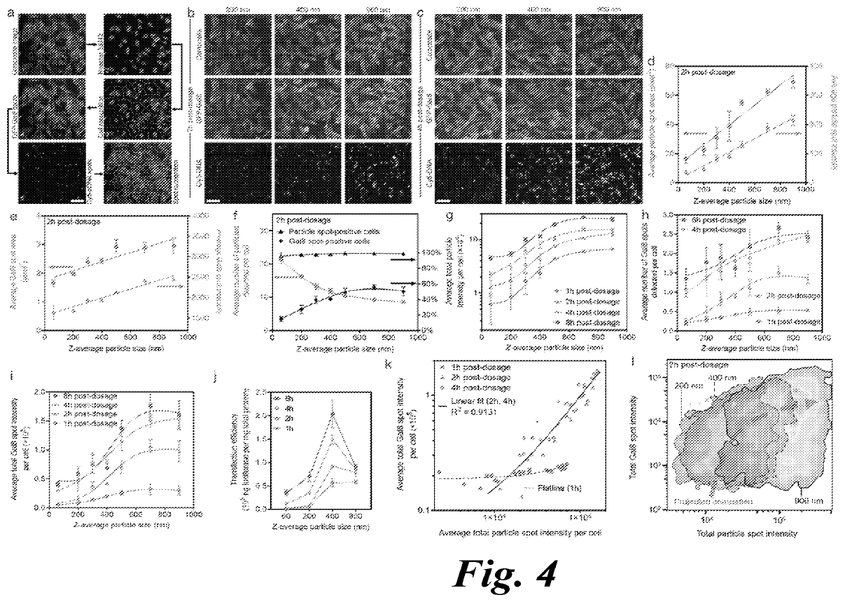

To assess cellular uptake and endosomal escape of the pDNA/PEI particles at different sizes, which are two major intracellular barriers for transfection, Lachelt and Wagner, 2015, pDNAs were labeled with Cy5 and used a genetically modified B16F10 cell line that expresses galectin-8 (Gal8) fused with GFP as the assessment tools. The Gal8 proteins that distributed throughout the cytosol bind to the cell membrane glycans exposed upon damage of endosomal vesicles, which subsequently aggregate and form GFP puncta (FIG. 4A). Thurston et al., 2012. Previous reports have suggested that the delivery efficiency of siRNA, Kilchrist et al., 2019, or CRISPR-Cas9 ribonucleo-proteins, Rui et al., 2019, by cationic polymers were positively correlated with the average number of Gal8 spots, i.e., successful endosomal escape events, detected per cell.

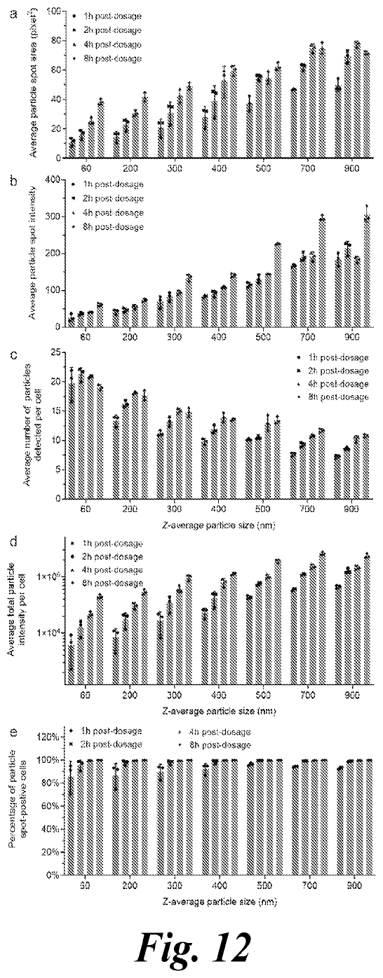

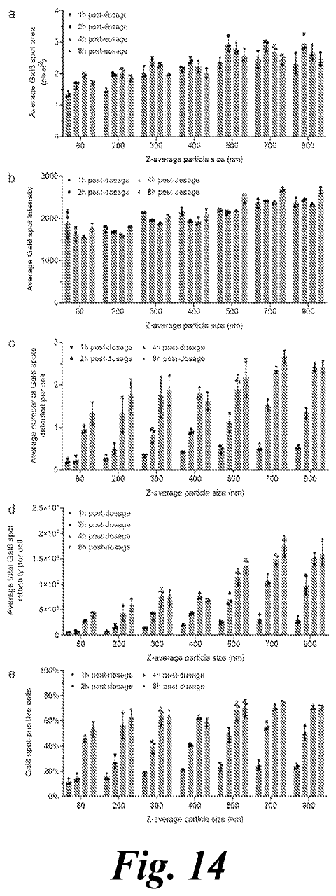

Both the particle uptake (Cy5 spots) and endosomal escape (GFP spots) were quantitatively analyzed by Cellomics high-content analysis (HCA) on fixed cells upon treatment of particles for 1, 2, 4 or 8 h (the full data panel is in FIG. 12, FIG. 14). The particles recognized showed a clear increasing trend in the area and intensity as the particle size increased (FIG. 4B-FIG. 4D and FIG. 10 and FIG. 11). Note that: The 2-h data points in FIG. 14A and FIG. 14B also are shown in FIG. 4E; All the data points in FIG. 14C are shown in FIG. 4H; All the data points in FIG. 14D are shown in FIG. 4I; The 2-h data points in FIG. 14E are shown in FIG. 4F.

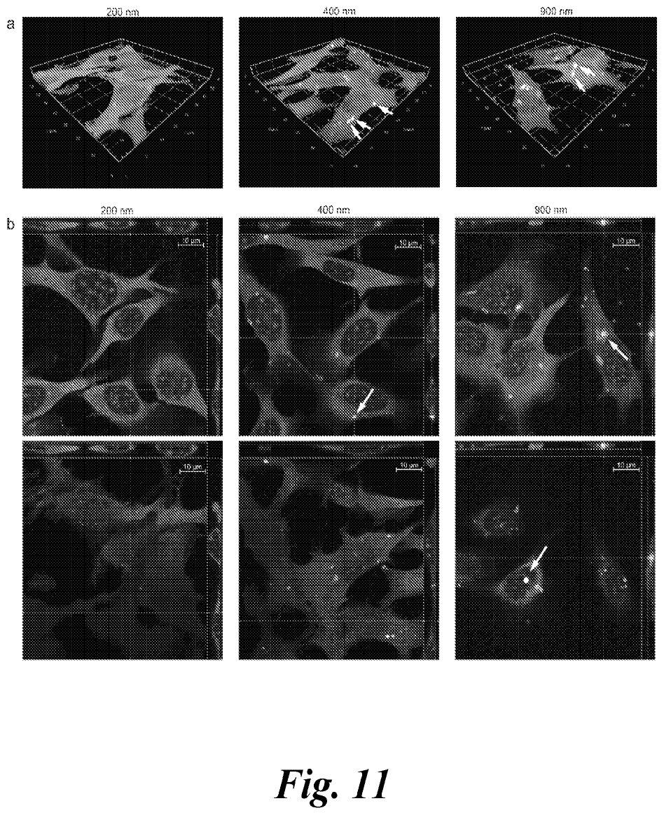

The observations by confocal laser scanning microscopy in FIG. 11 confirm the major findings from high-throughput Cellomics measurements that: (1) Particle size differences were clearly distinguished inside the cells among different groups, and larger particles induced larger endocytic vesicles suggested by larger Gal8-GFP puncta; (2) The level of endosomal escape scales with the overall uptake level, that a larger size inducing stronger endosomal escape.

It also is notable that: (1) The white arrows show overlap of the particle signals with the endosomal escape indicator Gal8-GFP puncta. This suggests that some particles induced endosomal rupture but are still associated with the damaged vesicle membranes; (2) The yellow arrow in 900-nm group shows satellite-distributed, small particles that were seemingly dissociated from a giant one that just escaped its endosome. The body of the giant particle is still associated with the damaged vesicle membranes; (3) For smaller particles, especially the 200-nm group, the particles concentrated near the basolateral side where the cells attach to the surface, as shown in the bottom panel for 200-nm and 400-nm groups in FIG. 11B. Such feature was not observed for 900-nm group. The implications from (2) and (3) are still illusive to us and are not the focus of this study;

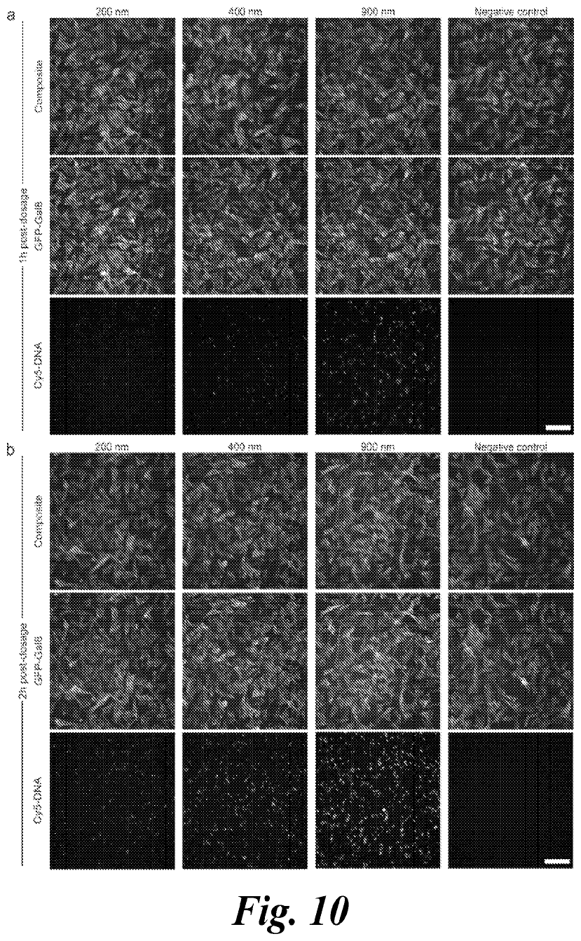

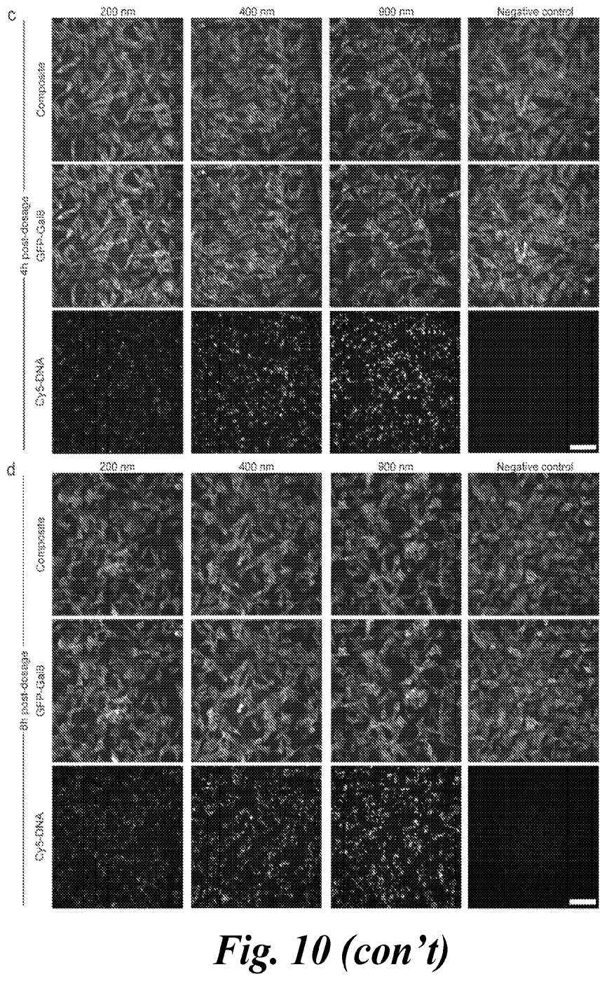

Note that the images shown in FIG. 10 are randomly selected from the pool of Cellomics-obtained images (1 out of 90 for each size group). To demonstrate the particle-cell interactions in greater details, a frame that is ¼ the original area of the image was created to include a representative region, and then enlarged 4 times to generate each image in FIG. 4B.

Note that the 2-h data points in FIG. 12A and FIG. 12B are shown in FIG. 4D; The 2-h data points in FIG. 12C are shown in FIG. 4F; All the data points in FIG. 12D are shown in main text FIG. 4G; The 2-h data points in FIG. 12E are shown in FIG. 4F.

FIG. 12A and FIG. 12B show the verification of the particle cellular uptake by pDNAs labeled with tritium. FIG. 12A, disintegration events per minute (DPM) detected in control samples (100 μL of suspension of stabilized particles with 0.5 μg pDNA) for particles at different sizes, showing no influence of particle size on the scintillation assay; FIG. 12B, absolute uptake measure of particles at different sizes after particle incubation for 2 or 4 h, relative to a total dosage of 0.1 μg pDNA per 10⁴ cells.

The method of ³H labeling of pDNA and assessments of absolute cellular pDNA uptake was described fully in previous reports. Hu et al., 2019; Williford et al., 2016. The use of this radioactive substrate tritium was approved by Johns Hopkins University Radiation Safety Office. Briefly, the pDNA was labeled by ³H through methylation reaction mediated by methyltransferase (New England BioLabs, USA) with the substrate of SAM[³H] (adenosyl-L-methionine, S-[methyl-³H]) (PerkinElmer, USA). The pDNA was then subjected to column washing using a standard QIAprep Spin Miniprep pDNA purification kit (Qiagen, USA). The labeled pDNA was blended with unlabeled pDNA before formulation of particles and dosage to the cells as described in the main text. At 1 or 2 h post-dosage, the transfection medium containing the particles were drained, followed by intense washing of heparin-containing PBS (100 IU mL⁻¹, to remove surface-bound particles) and fresh PBS. The cells were lysed by 2 freeze-thaw cycles in reporter lysis buffer, with the lysate mixed with an equal volume of SOLVABLE solution (PerkinElmer, USA). The SOLVABLE solution solubilized ³H labeled nucleotides that gained access to Ultima Gold scintillation fluid (PerkinElmer, USA) added subsequently. The radioactivity (disintegration per minute, DPM, a quantitative measure of the absolute ³H amount) was assessed by a Tri-Carb 2200CA liquid scintillation analyzer (Packard Instrument Company, USA).

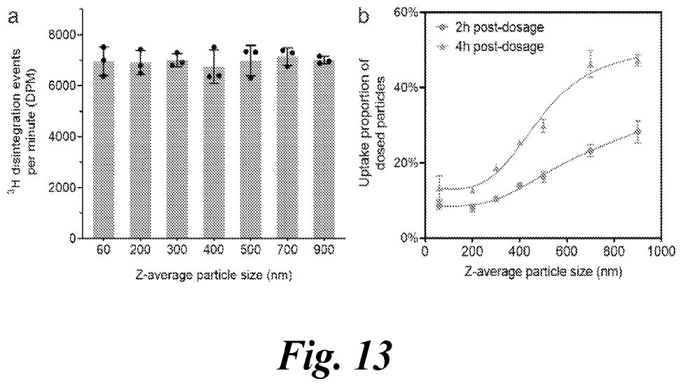

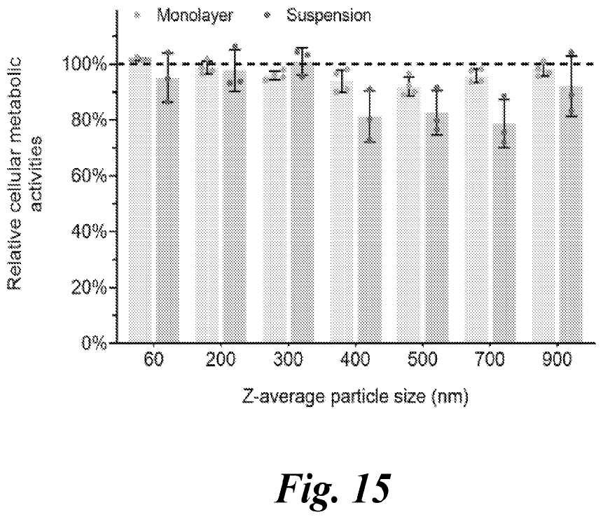

Larger particles induced Gal8 spots with increasing average area and intensity (FIG. 4E), indicating higher surface area for Gal8 binding thus formation of larger endocytic vesicles prior to escape events. Considering the size range of these pDNA/PEI particles (up to 900 nm) exceeds the typical sizes acceptable for clathrin-dependent or independent endocytosis pathways, Rejman et al., 2004; Mayor and Pagano, 2007, uptake of these particles was more likely through a phagocytosis-like pathway. Kopatz et al., 2004. The drop of particle number per cell as size increased (FIG. 4F) was as predicted, given the decreasing particle number concentration as a result of increasing pDNA payload per particle. Nonetheless, the fewer larger particles yielded much higher efficiency in endosomal escape, presented as a higher percentage of Gal8 spot-positive cells (FIG. 4F). It is also worth noting that the Gal8 spot-positive percentage largely matched the transgene expression-positive percentage (FIG. 3B). There was a sharp increase in the total uptake amount (measured by average total particle intensity per cell, and another quantitative ³H-labeled DNA assay described in FIG. 13) at all time points in the size range of 60 nm to 500 nm, and a slight increase as particle size increased from 500 nm to 900 nm (FIG. 4G). The average Gal8 spot number per cell for each group was measured to assess the overall endosomal escape level, which was reported to strongly correlated with transfection efficiency (FIG. 4H). Kilchrist et al., 2019; Rui et al., 2019. Considering the differences observed in Gal8 spot area and intensity (FIG. 4E), the average total Gal8 spot intensity per cell gives a better assessment of the overall endosomal escape level (FIG. 4I). In addition, the kinetics of the uptake and endosomal escape matched the kinetics of luciferase expression following particle incubation with different lengths (FIG. 4J). The reasons for a drop in transfection efficiency mediated by particles with a size larger than 400-nm (HEK293T) or 500-nm (HEK293F), even though they showed higher degrees of particle uptake and endosomal escape, remain to be elucidated in future work; although it was not due to any change in cellular metabolism activities (FIG. 15).

For the data in FIG. 15, the alamarBlue reagent (Thermo Fisher Scientific, USA) was added to HEK293T cells after they were incubated with the pDNA/PEI particles for 4 h in monolayer culture or added to HEK293F cells together with the pDNA/PEI particles. The assay reagent stayed in the medium for 20 h for monolayer culture and 48 h for suspension culture. The 100% reference level was derived from control cells treated with particle-free transfection medium. An absorbance-based assay at the wavelengths of 570 nm and 600 nm was conducted to 100 μL of final media following the protocol from the manufacturer.

The results showed that: (1) Even though uptake increased with larger size of the particles, there was no associated reduction in cellular metabolic activities. This suggested that the reduction in transfection efficiency seen from 400/500 nm to 900 nm could not be explained by potential cytotoxicity due to higher uptake levels of particles and PEI; (2) Slight reduction in metabolic activities was seen with particle sizes that induced the highest transfection efficiency: 400 and 500 nm for monolayer culture and 400 nm, 500 nm, and 700 nm for suspension culture. The indication of this observation is currently unknown.

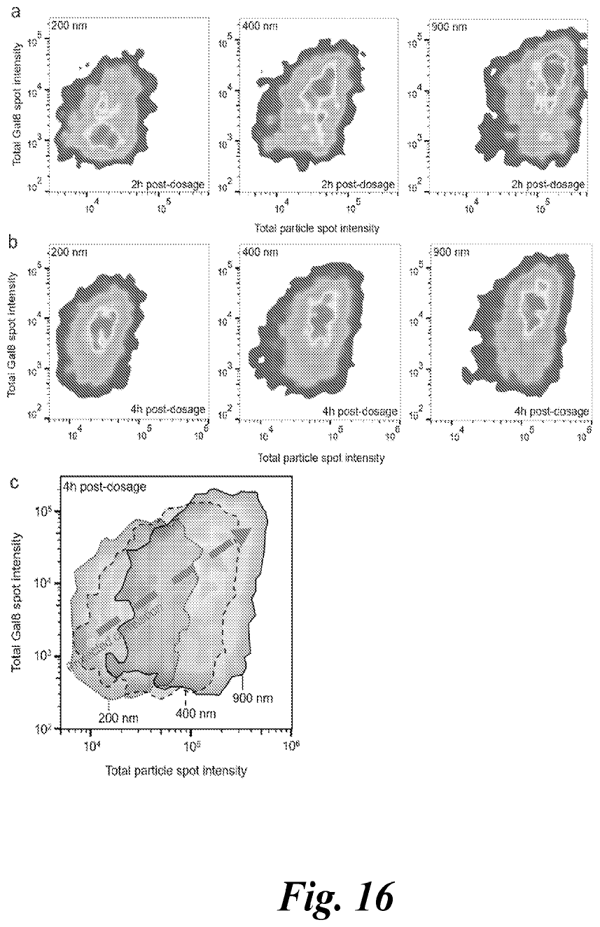

For pDNA/PEI particle-mediated transfection, the relationships between cellular uptake and endosomal escape on a plate well-average (FIG. 4K) or single-cell level (FIG. 4L) were analyzed. Endosomal escape events mainly took place after 1 h of incubation with the particles. A single linear regression was shared by all data points of the plate well-average readings (FIG. 4K) at 2 h and 4 h post-dosing, regardless of the particle size. There was a positive correlation that lines all the areas with the highest cell density on the heat maps across different particle sizes on the single-cell level (FIG. 4L and FIG. 16, plotted using FACS files exported by Cellomics and processed by FlowJo). These analyses demonstrate that the endosomal escape degree scales with the cellular uptake degree of the pDNA/PEI particles, and the transfection efficiency observed was controlled by a size-dependent mechanism limited by cellular uptake.

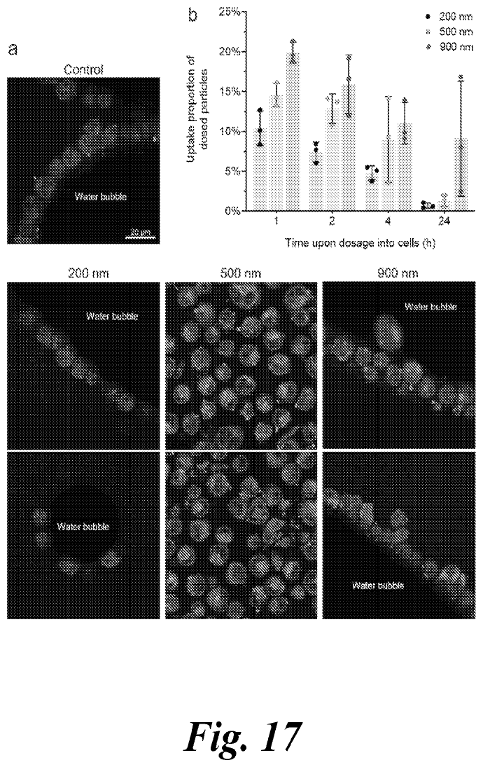

In suspension culture of HEK293F cells, the cellular uptake of particles with different sizes was found to be consistent with the findings in monolayer culture of HEK293T or B16F10-GFP-Gal8 cells, as quantified by the ³H-DNA assay and directly observed by confocal laser scanning microscopy (FIG. 17). This finding partially explained the similarity seen in the transfection activities of the particles at different sizes (FIG. 3).

It is remarkable that a suspension culture of HEK293F cells showed very similar results as a monolayer culture (FIG. 10, FIG. 11): (1) The particles in the cell bodies showed distinct sizes as per their relatively controlled sizes (FIG. 17A), proving again that the diluted nature in the transfection medium kinetically limited the growth of particles and preserved their size (FIG. 9), and it is important to control the size before dosage; (2) The uptake positively correlated with the particle size, as qualitatively illustrated by the images and quantitatively proved by ³H-labeling assay (the methods are in the associated notes under FIG. 13). This assay further revealed that the intracellular pDNA quantity peaked within 1 h upon dosage and decreased as the culture continued, presenting drastic differences in uptake kinetics as a monolayer culture. This might be a result from the intense mixing conditions in the suspension culture that enabled rapid cell-particle contact shortly after dosage. Under such conditions, larger particles still exhibited greater uptake rate. These findings enhanced the view of that particle size control is the key for optimization of the transient transfection process for production of lentiviral vectors.

### 1.2.5 Scaled Manufacturing of Concentrated 400-nm Particles and Product Validation for LVV Production

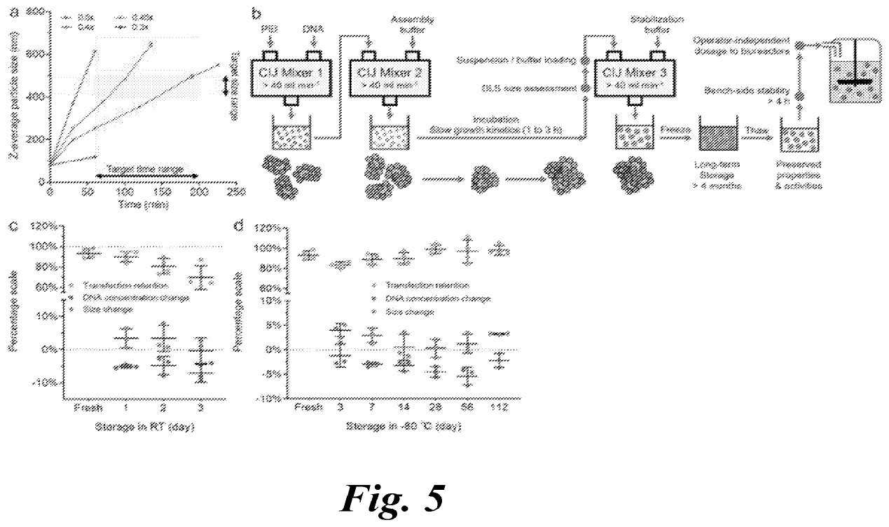

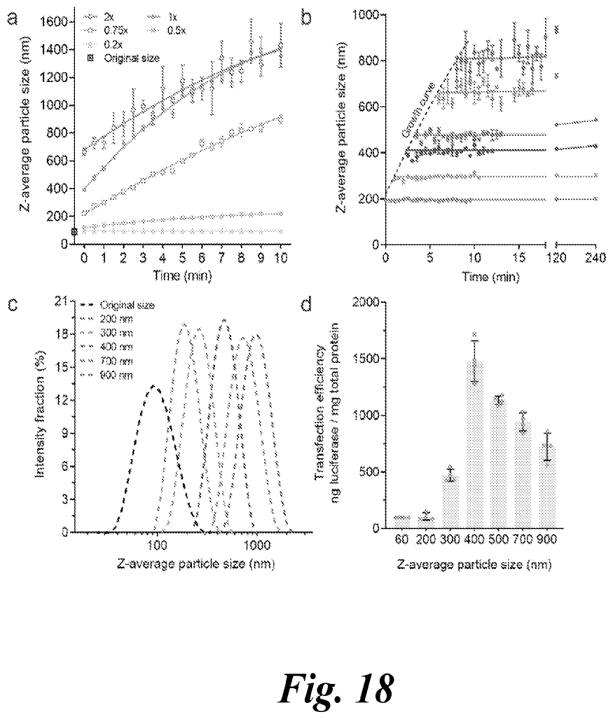

The particle assembly process could be scaled up by implementing the two mixing steps (particle growth and stabilization) with relatively high flow rates (e.g., 40 mL min⁻¹) in CIJ devices. As it still takes appreciable time to generate a high volume of particles required for large bioreactors, the fast growth kinetics shown in FIG. 2C was impractical. To address this, the pDNA concentration was doubled to reduce the volume requirement, and verified size control, stability and transfection efficiency of the particles (FIG. 18). The ionic strength of the medium was optimized for the particle growth step to control the particle growth rate within 1 to 3 h for the ease of operation (FIG. 5A). As streamlined in FIG. 5B, given the extended particle growth time, the time required to flow growing particles out of the CIJ mixer #2 and to load solutions into the CIJ mixer #3 was insignificant. This makes the whole process operator-independent, reproducible, and less sensitive to the exact timing for incubation. The lowered ionic strength for the particle growth medium, and the consequently lower amount of acid required to revert the pH in the stabilization step, provided additional benefits on maintaining colloidal stability of the particles and preservation of transfection activities in storage. The particles were stable for 2 days in the suspension form at ambient temperature (FIG. 5C), and for more than 4 months when stored at −80° C. (FIG. 5D). Upon thawing from the samples at different time points during the storage period, the pDNA/PEI particles exhibited insignificant changes in physical properties and transfection efficiency.

### 1.3 Experimental Section

### 1.3.1 Cell Cultures and Transfection Studies

For monolayer culture studies, HEK293T cells (American Type Culture Collection, USA; maintained in DMEM+10% FBS and 2 mM L-glutamine, at 37° C., 5% CO₂, and saturated humidity) were seeded into 24-well plates at a cell density of 25,000 cells well⁻¹ 1 day prior to transfection. The particles were pipetted into FreeStyle 293 medium in 5-ml microcentrifuge tubes, immediately followed by vortex for 10 sec to reach a final particle concentration of 1 μg pDNA mL⁻¹. For example, 100 μL of a particle suspension at 25 μg pDNA mL⁻¹ was pipetted into 2.4 ml of serum-free FreeStyle 293 medium. The original medium in the wells was then drained and replaced by 500 μL of the particle-containing medium. At 4 h post-dosing, the medium was replaced by fresh full medium. A 20-h incubation was followed to allow transgene expression. For suspension culture studies, HEK293F cells (Thermo Fisher Scientific, USA; maintained in FreeStyle 293 medium, at 37° C., 8% CO₂, and saturated humidity) were seeded into a 12-well plate equipped with a SpinS™ Bioreactors plate spinner (3Dnamics, USA) at a cell density of 0.5×10⁶ cells mL⁻¹ at 1 day prior to transfection. The spinner was motorized at a rate of 150 rounds per minute for the duration of the experiments. The particles were pipetted into the wells all at once, followed by brief shaking of the plate, giving a final particle concentration of 1 μg pDNA mL⁻¹. For example, 80 μL of a particle suspension at 25 μg pDNA mL⁻¹ was pipetted into 2 mL of the cell suspension within a single well. When a whole plate was finished, the spinner was reconnected. A 48-h incubation was followed to reach the peak transgene expression. When characterizing luciferase as the reporter, the cells were lysed by reporter lysis buffer (Promega, USA) using two freeze-thaw cycles, with the lysate characterized by a luminometer upon addition of luciferin assay solution (Promega, USA) against a ladder generated by the standardized luciferase samples (Promega, USA). When characterizing GFP as the reporter, the cells were suspended by trypsin-EDTA in PBS supplemented with 1% FBS and 0.5 mM EDTA and analyzed by a FACSCanto flow cytometer (BD Life Sciences, USA).

### 1.3.2 Assembly of Stable Particles at Different Sizes

The pDNA/PEI nanoparticles as the building blocks were first synthesized based on previous reports. Santos et al., 2016; Hu et al., 2019. Briefly, pDNAs (multiple species with gWiz-Luc or gWiz-GFP from Aldevron, USA as a reporter) and PEIpre (Polyplus, France) were separately dissolved in ultrapure water, then pumped into a confined impinging jet (CIJ) mixer, Johnson and Prud'homme, 2003; Hao et al., 2020, at a flow rate of 20 mL min 1. The concentration was either 100 μg pDNA mL⁻¹ (FIG. 2, generating 60 to 70-nm nanoparticles) or 200 μg pDNA mL⁻¹ (FIG. 5, generating 80 to 100-nm nanoparticles); and the N/P ratio was 5.5. For small-batch preparations (FIG. 2-4), 100 μL of PBS solution (from 0.2× to 2×) was pipetted into 100 μL of the 60-nm nanoparticle suspension, immediately followed by 3 sec of vortex. At different time points (shown in FIG. 2B), 200 μL of the 20 mM HCl in 19% (w/w) trehalose solution was pipetted into the growing particles, immediately followed by 3 sec of vortex. The particles were stabilized and ready for use and characterization.

For large-scale productions of the pDNA/PEI particles (FIG. 5), the 80- to 100-nm nanoparticles and PBS (0.4× or 0.45×) were loaded in syringes separately and pumped into a CIJ device at a flow rate of 20 mL min⁻¹. The eluate was collected and incubated under room temperature without stirring. Dynamic light scattering (Zetasizer ZS90, Malvern, USA) was conducted periodically to monitor the size. When particles reached the target size, the particle suspension, and the solution of 2.5 mM HCl in 19% (w/w) trehalose were loaded in syringes separately and pumped into the CIJ device at a flow rate of 20 mL min⁻¹. The particles were ready for use or freezing down to −80° C. for long-term storage. For transfection experiments, the frozen particles were retrieved by thawing at ambient temperature followed by a brief vortex. The particles were then ready for use or temporarily stored for up to 1-2 days at ambient temperature without compromising transfection activity.

### 1.3.3 Quantitative Cellular Uptake and Endosomal Escape Assessments by Cellomics

pDNA was labeled by covalently linking Cy5-amine (Lumiprobe, USA) to pDNA via UV-induced crosslinking of NHS-psoralen (Thermo Fisher Scientific, USA). Wilson et al., 2017. The Cy5-labeled pDNA was blended into the pDNA mixture at 5% prior to particle formulation. B16F10 cell line expressing GFP-coupled galectin-8 (GFP-Gal8) was obtained by transfection using plasmids encoding Super PiggyBac Transposase (System Biosciences, USA) and Piggybac-transposon-GFP-Gal8 (Addgene plasmid #127191) and a poly(beta-amino ester) (PBAE) carrier, Karlsson et al., 2020, then sorted by a SH800 cell sorter (Sony, Japan) twice. The cells were cultured in DMEM supplemented with 10% FBS at 100,000 cells per well. The particles were dosed 24 h later as described above, except the transfection medium was switched to Opti-MEM for optimal results in this cell line. After incubation of predetermined times, cells were washed by PBS for three times, fixed by 4% paraformaldehyde (PFA) solution, stained by Hoechst 33342, and then washed by PBS for three times.

The plates were analyzed by a CellInsight CX7 High-content Analysis (HCA) platform (Thermo Fisher Scientific, USA). A brief example of the analysis process is given in FIG. 4A. Imaging was conducted at 20× magnification with a resolution of 1104×1104 pixel per field correlating with an area of 501.2×501.2 μm². A total of 30 fields were analyzed inside each well of the plates, and the well-averaged result was generated by averaging all the cells in all the fields. The <SpotDetector.V4> program was used as supplied by the manufacturer with laser/filter sets of Channel 1: 386/440 nm, Channel 2: 485/521 nm, and Channel 3: 650/694 nm with fixed exposure times. In the analysis, the identifications of cell nuclei, GFP-Gal8 spots and Cy5-pDNA spots were carried out with appropriate smoothing and thresholding settings that were verified by eye to obtain correct recognitions in sample images. In thresholding, <Isodata> (comparing each pixel with its surrounding) was used for GFP-Gal8 due to its high background (cytosolic Gal8); while <Fixed> (setting a predetermined level) was used for Cy5-pDNA due to its clean background, potentially irregular shapes, and large areas. Cell body (cytosolic area) identification and segmentation were approximated by extending the area attributed from each identified nucleus outward by 30 pixels (13.6 μm) with no overlap between adjacent cells (FIG. 4A).

### 1.4 Summary

This study revealed the key insight that the transfection efficiency in LVV production cell lines was critically dependent on the size of pDNA/PEI particles and identified 400 nm to 500 nm as the optimal size range for transfection. A stepwise process was designed based on surface charge inversion and conditioning of ionic strength, and pDNA/PEI particles with an average size of 60 nm to 1000 nm were prepared with a high degree of size control. The prepared particles exhibited excellent stability in suspension at ambient temperature for standard operations and at −80° C. for long-term storage. This particle size engineering method confers high uniformity, and the sequential steps permits high tunability of the assembly kinetics. A scale-up production method was developed based on a continuous flow mixing process—the FNC platform—with a tailored assembly kinetics to accommodate the mixing procedure. The optimal transfection activity and stability of the 400-nm pDNA/PEI particle formulation was validated in production of LVVs using pre-prepared, freeze-stored, transported, and thawed particles, showing matching performance with the particles produced using the industry standard in realistic bioreactor settings. This new scalable manufacturing method has high translational potential that can be easily extended to production of a wide range of gene therapy vectors with improved productivity and quantity control.

### Example 2

### Comparative Example—Scale-Up Production of Plasmid DNA/Polycation Particles with Defined Size

### 2.1 Experimental Set-Up

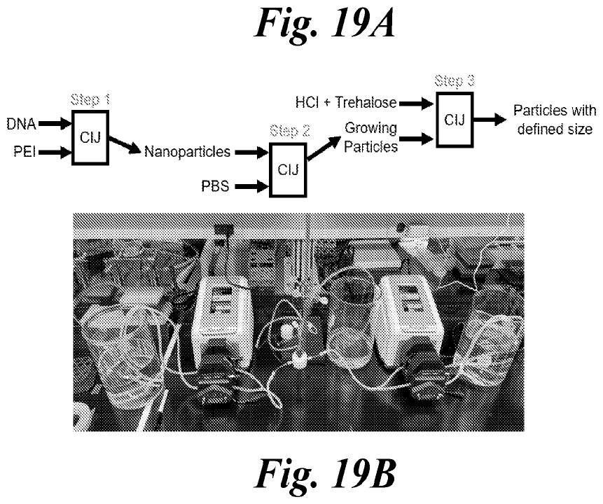

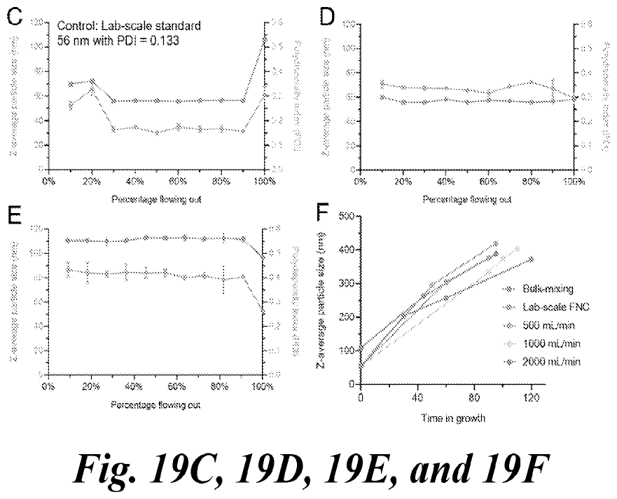

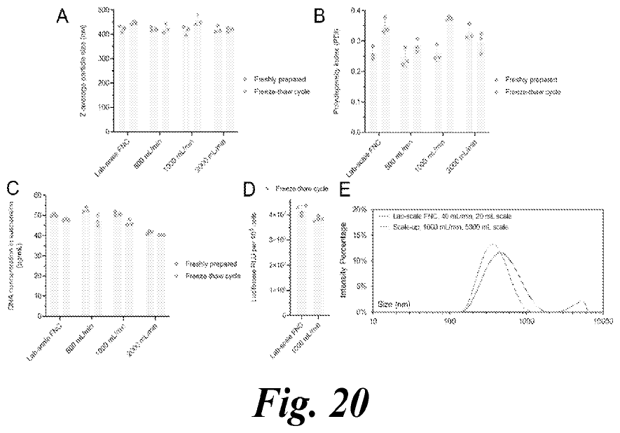

Plasmid DNA (4.4 kb) was dissolved in ultra-pure water at a concentration of 400 μg/mL; The polycation, i.e., poly(ethyleneimine) (in vivo-jetPEI from Polyplus, Inc.) was dissolved in ultra-pure water at a concentration of 317.6 μg/mL that was equivalent to a nitrogen-to-phosphate ratio of 6. A typical formulation process to obtain particles with defined sizes is shown in FIG. 19A and involves three steps: Step 1, complexation; Step 2, size growth; and Step 3, stabilization. For the three experimental groups with operational flow rate of 500 mL/min, 1000 mL/min, or 2000 mL/min, an enlarged confined impinging jet (CIJ) mixer was used, with each inlet extracting solution from a reservoir driven by a peristaltic pump (FIG. 19B). For Step 1, the plasmid DNA and PEI solutions were loaded and pumped into the CIJ mixer, generating stable nanoparticles. Nanoparticles were collected directly into aliquots as portions of the total flow volume to evaluate the steadiness of complexation (FIG. 1C, FIG. 1D, FIG. 1E). For Step 2, the nanoparticle suspension and a solution of 0.44-fold phosphate buffered saline (PBS) were loaded and pumped into the CIJ mixer, generating growing particles. For Step 3, when the size of the growing particles reached the target of 400 nm, the growing particle suspension and a solution of 2.5 mM HCl plus 19% (w/w) trehalose solution were loaded and pumped into the CIJ mixer, generating stabilized particles with defined 400 nm size.

For transfection tests, HEK293T cells were seeded at 100,000 cells/well in 24-well plate, 1 day prior to particle dosage. Stabilized particles (out of Step 3, at a DNA concentration of 50 μg/mL, containing 5% luciferase plasmid) were diluted by Opti-MEM medium to a DNA concentration of 1 μg/mL. Cells were incubated in particle-containing medium for 4 h, followed by culture in full medium for 20 h.

### 2.2 Description

For Step 1, a standard lab-scale setting generated nanoparticles with a z-average diameter of 56 nm and a polydispersity index (PDI) of 0.133. Using the peristaltic pump and reservoir-based set up, at a flow rate of 500 mL/min, the same size and a similar PDI were obtained when the flow is steady (FIG. 1C). When the flow rate was increased to 1000 mL/min, nanoparticles with the same size but elevated PDI (between 0.3 and 0.4) were obtained (FIG. 19D). When the flow rate was further increased to 2000 mL/min, nanoparticles had a nearly doubled size (around 150 nm) and an elevated PDI (around 0.4), though the nanoparticle quality appeared to be steady during the entire flowing process (FIG. 1E). The increased size and/or PDI of the nanoparticles, however, did not have a significant impact on the growth kinetics of Step 2, as all flow rates had similar growth profiles as compared to standard lab-scale operation (FIG. 1F).

TABLE 1
Volume specifications for test experiments with different scales.

| | | Complexation | | Size growth | | Stabilization | | |
|---|---|---|---|---|---|---|---|---|
| Experiment | | (Step 1) | | (Step 2) | | (Step 3) | | Equipment |
| Lab-scale | | 5 | mL | 7.5 | mL | 10 | mL | Single |
| 40 | mL/min | | | | | | | syringe |
| | | | | | | | | pump |
| 500 | mL/min | 100 | mL | 170 | mL | 300 | mL | Peristaltic |
| | | | | | | | | pump + |
| | | | | | | | | reservoir |
| 1000 | mL/min | 750 | mL | 1400 | mL | 2650 | mL | Peristaltic |
| | | | | | | | | pump + |
| | | | | | | | | reservoir |
| 2000 | mL/min | 250 | mL | 450 | mL | 800 | mL | Peristaltic |
| | | | | | | | | pump + |
| | | | | | | | | reservoir |

* The specifications in this table represent the volume loaded for each inlet into the confined impinging jet mixer.

Upon Step 3, all preparations with different flow rates generated particles with the target defined size of 400 nm (FIG. 20A) and a similar PDI (FIG. 20B). Nanodrop assessment of the DNA concentration (with a target of 50 vg/mL) in the suspension revealed that 500 mL/min and 1000 mL/min did not generate any loss during the formulation processes, while 2000 mL/min resulted in a slight loss (around 5 μg/mL, FIG. 20C). These characteristics were preserved upon a cycle of freezing (to minus 80 degree Celsius) and thawing (FIG. 20A-20C). 5% luciferase plasmids were added to the formulations prepared by the standard lab-scale flow rate of 40 mL/min or scale-up flow rate of 1000 mL/min, and in vitro transfection assay on HEK293T cells demonstrated similar transfection efficiency (FIG. 20D). These two formulations also had a similar particle size distribution as assessed by dynamic light scattering (FIG. 20E). These data collectively demonstrate that the formulation method as shown in FIG. 19A to generate plasmid DNA/PEI particles with defined size is scalable.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art. In case of a conflict between the specification and any of the incorporated references, the specification (including any amendments thereof, which may be based on an incorporated reference), shall control. Standard art-accepted meanings of terms are used herein unless indicated otherwise. Standard abbreviations for various terms are used herein.
Milone, M. C. and O'Doherty, U. Clinical use of lentiviral vectors. Leukemia 32, 1529-1541 (2018).
Wang, D., Tai, P. W. L. and Gao, G. Adeno-associated virus vector as a platform for gene therapy delivery. Nature Reviews Drug Discovery 18, 358-378 (2019).
Merten, O.-W., Hebben, M. and Bovolenta, C. Production of lentiviral vectors. Molecular Therapy—Methods and Clinical Development 3, 16017 (2016).
Pear, W. S., Nolan, G. P., Scott, M. L. and Baltimore, D. Production of high-titer helper-free retroviruses by transient transfection. Proceedings of the National Academy of Sciences 90, 8392-8396 (1993).
Dalby, B. et al. Advanced transfection with Lipofectamine 2000 reagent: primary neurons, siRNA, and high-throughput applications. Methods 33, 95-103 (2004).
Boussif, O. et al. A versatile vector for gene and oligonucleotide transfer into cells in culture and in vivo: polyethylenimine. Proceedings of the National Academy of Sciences 92, 7297-7301 (1995).
Mislick, K. A. and Baldeschwieler, J. D. Evidence for the role of proteoglycans in cation-mediated gene transfer. Proceedings of the National Academy of Sciences 93, 12349-12354 (1996).
Rejman, J., Bragonzi, A. and Conese, M. Role of clathrin- and caveolae-mediated endocytosis in gene transfer mediated by lipo- and polyplexes. Molecular Therapy 12, 468-474 (2005).
Bus, T., Traeger, A. and Schubert, U. S. The great escape: how cationic polyplexes overcome the endosomal barrier. Journal of Materials Chemistry B 6, 6904-6918 (2018).
Pollard, H. et al. Polyethylenimine but Not Cationic Lipids Promotes Transgene Delivery to the Nucleus in Mammalian Cells. Journal of Biological Chemistry 273, 7507-7511 (1998).
van der Loo, J. C. M. and Wright, J. F. Progress and challenges in viral vector manufacturing. Human Molecular Genetics 25, R42-R52 (2015).
Santos, J. L. et al. Continuous Production of Discrete Plasmid DNA-Polycation Nanoparticles Using Flash Nanocomplexation. Small 12, 6214-6222 (2016).
Hu, Y. et al. Kinetic Control in Assembly of Plasmid DNA/Polycation Complex Nanoparticles. ACS Nano 13, 10161-10178 (2019).
Ogris, M. et al. The size of DNA/transferrin-PEI complexes is an important factor for gene expression in cultured cells. Gene Therapy 5, 1425-1433 (1998).
Zhang, W. et al. Nano-Structural Effects on Gene Transfection: Large, Botryoid-Shaped Nanoparticles Enhance DNA Delivery via Macropinocytosis and Effective Dissociation. Theranostics 9, 1580-1598 (2019).
Tockary, T. A. et al. Single-Stranded DNA-Packaged Polyplex Micelle as Adeno-Associated-Virus-Inspired Compact Vector to Systemically Target Stroma-Rich Pancreatic Cancer. ACS Nano 13, 12732-12742 (2019).
Osada, K. et al. Quantized Folding of Plasmid DNA Condensed with Block Catiomer into Characteristic Rod Structures Promoting Transgene Efficacy. Journal of the American Chemical Society 132, 12343-12348 (2010).
Curtis, K. A. et al. Unusual Salt and pH Induced Changes in Polyethylenimine Solutions. PLOS ONE 11, e0158147 (2016).
Bertschinger, M. et al. Disassembly of polyethylenimine-DNA particles in vitro: Implications for polyethylenimine-mediated DNA delivery. Journal of Controlled Release 116, 96-104 (2006).
Johnson, B. K. and Prud′homme, R. K. Chemical processing and micromixing in confined impinging jets. AIChE Journal 49, 2264-2282 (2003).
Hao, Y., Seo, J.-H., Hu, Y., Mao, H.-Q. and Mittal, R. Flow physics and mixing quality in a confined impinging jet mixer. AIP Advances 10, 045105 (2020).
Bertschinger, M., Chaboche, S., Jordan, M. and Wurm, F. M. A spectrophotometric assay for the quantification of polyethylenimine in DNA nanoparticles. Analytical Biochemistry 334, 196-198 (2004).
Lächelt, U. and Wagner, E. Nucleic Acid Therapeutics Using Polyplexes: A Journey of 50 Years (and Beyond). Chemical Reviews 115, 11043-11078 (2015).
Thurston, T. L. M., Wandel, M. P., von Muhlinen, N., Foeglein, A. and Randow, F. Galectin 8 targets damaged vesicles for autophagy to defend cells against bacterial invasion. Nature 482, 414-418 (2012).
Kilchrist, K. V. et al. Gal8 Visualization of Endosome Disruption Predicts Carrier-Mediated Biologic Drug Intracellular Bioavailability. ACS Nano 13, 1136-1152 (2019).
Rui, Y. et al. Carboxylated branched poly((3-amino ester) nanoparticles enable robust cytosolic protein delivery and CRISPR-Cas9 gene editing. Science Advances 5, eaay3255 (2019).
Rejman, J., Oberle, V., Zuhorn, I. S. and Hoekstra, D. Size-dependent internalization of particles via the pathways of clathrin- and caveolae-mediated endocytosis. Biochemical Journal 377, 159-169 (2004).
Mayor, S. and Pagano, R. E. Pathways of clathrin-independent endocytosis. Nature Reviews Molecular Cell Biology 8, 603-612 (2007).
Kopatz, I., Remy, J.-S. and Behr, J.-P. A model for non-viral gene delivery: through syndecan adhesion molecules and powered by actin. The Journal of Gene Medicine 6, 769-776 (2004).
Wilson, D. R. et al. A Triple-Fluorophore-Labeled Nucleic Acid pH Nanosensor to Investigate Non-viral Gene Delivery. Molecular Therapy 25, 1697-1709 (2017).
Karlsson, J., Rhodes, K. R., Green, J. J. and Tzeng, S. Y. Poly(beta-amino ester)s as gene delivery vehicles: challenges and opportunities. Expert Opinion on Drug Delivery 17, 1395-1410 (2020).
Yue, Y. et al. Revisit complexation between DNA and polyethylenimine—Effect of uncomplexed chains free in the solution mixture on gene transfection. Journal of Controlled Release 155, 67-76 (2011).
Ohi, M., Li, Y., Cheng, Y. and Walz, T. Negative staining and image classification—powerful tools in modern electron microscopy. Biological Procedures Online 6, 23-34 (2004).
Williford, J.-M. et al. Critical Length of PEG Grafts on lPEI/DNA Nanoparticles for Efficient in Vivo Delivery. ACS Biomaterials Science and Engineering 2, 567-578 (2016).

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A method for preparing a plurality of polycation/polyanion complex nanoparticles, the method comprising:
(a) flowing a first stream comprising one or more water-soluble polycationic polymers at a first variable flow rate and a second stream comprising one or more polyanionic polymers at a second variable flow rate into a first flash nanocomplexation (FNC) mixer to form a plurality of nanoparticles having a first particle size;
(b) flowing a third stream comprising the plurality of nanoparticles having a first particle size at a third variable flow rate and a fourth stream comprising an assembly buffer at a fourth variable flow rate into a second FNC mixer to form a plurality of assembled nanoparticles;
(c) incubating the plurality of assembled nanoparticles formed in step (b) for a period of time to form a plurality of assembled nanoparticles having a second particle size; and
(d) flowing a fifth stream comprising the plurality of assembled nanoparticles having a second particle size at a fifth variable flow rate and a sixth stream comprising a stabilization buffer at a sixth variable flow rate into a third FNC mixer to form a plurality of polycation/polyanion complex nanoparticles.

2. The method of claim 1, wherein the one or more water-soluble polycationic polymers are selected from the group consisting of polyethylenimine (PEI), chitosan, PAMAM dendrimers, protamine, poly(arginine), poly(lysine), poly(beta-aminoesters), cationic peptides and derivatives thereof.

3. The method of claim 1 or 2, wherein the one or more water-soluble polycationic polymers is polyethylenimine.

4. The method of claim 1 or 2, wherein the one or more water-soluble polyanionic polymers are selected from the group consisting of poly(aspartic acid), poly(glutamic acid), negatively charged block copolymers, heparin sulfate, dextran sulfate, hyaluronic acid, alginate, tripolyphosphate (TPP), oligo(glutamic acid), a cytokine, a protein, a peptide, a growth factor, and one or more nucleic acids.

5. The method of claim 4, wherein the one or more nucleic acids are selected from the group consisting of an antisense oligonucleotide, cDNA, genomic DNA, guide RNA, plasmid DNA, vector DNA, mRNA, miRNA, piRNA, shRNA, and siRNA.

6. The method of claim 5, wherein the one or more nucleic acids comprise plasmid DNA or a mixture of different species of one or more plasmid DNAs.

7. The method of claim 5, wherein the one or more nucleic acid comprise mRNA or a mixture of different species of one or more mRNAs.

8. The method of any of claims 1 to 7, wherein the first variable flow rate, the second variable flow rate, the third variable flow rate, the fourth variable flow rate, the fifth variable flow rate, and the sixth variable flow rate are each independently about 5 to 400 mL/min.

9. The method of any of claims 1 to 8, wherein the first particle size has an intensity average range between about 40 nm to about 120 nm.

10. The method of any of claims 1 to 9, wherein the plurality of nanoparticles having a first particle size are formed under conditions at a pH of about 2.0 to 4.0 and a conductivity of about 0.05 to 2.0 mS cm⁻¹.

11. The method of any of claims 1 to 10, wherein the plurality of nanoparticles formed in step (b) are incubated at about room temperature (22±4° C.) for a period of about 0.2 to about 5 hours.

12. The method of any of claims 1 to 11, wherein the plurality of assembled nanoparticles having a second particle size are formed under conditions at a pH of about 6.0 to 8.0, and a conductivity of about 2.0 to 25.0 mS cm⁻¹.

13. The method of any of claims 1 to 12, wherein the assembly buffer comprises phosphate buffered saline.

14. The method of claim 13, wherein the phosphate buffered saline comprises one or more of NaCl, KCl, Na₂HPO₄, KH₂PO₄, and combinations thereof.

15. The method of any of claims 1 to 14, wherein the second particle size has a range between about 300 nm to about 500 nm.

16. The method of any of claims 1 to 15, wherein the plurality of polycation/polyanion complex nanoparticles of step (d) are formed under conditions at a pH of about 2.0 to 4.0, and a conductivity of about 1.0 to 15.0 mS cm⁻¹.

17. The method of any of claims 1 to 16, wherein the stabilization buffer comprises at least one sugar.

18. The method of any of claims 1 to 17, wherein the one or more sugars comprise trehalose.

19. The method of claim 18, wherein the one or more sugars comprise between about 10% to about 30% w/w of trehalose.

20. The method of any of claims 1 to 19, wherein the stabilization buffer comprises HCl.

21. The method of any of claims 1 to 20, further comprising lyophilizing or freezing the particles at about −80° C. for storage.

22. A plurality of polycationic/nucleic acid nanoparticles comprising about 67±5 w/w % DNA; 9±5 w/w % bound polyethylenimine (PEI); and 24±5 w/w % residual polyethylenimine (PEI).

23. The plurality of polycationic/nucleic acid nanoparticles of claim 22, wherein the average zeta potential is about 35±5 mV.

24. The plurality of polycationic/nucleic acid nanoparticles of claims 22-23, wherein the plurality of polycationic/nucleic acid nanoparticles has a particle size ranging from about 300 nm to about 500 nm.

25. The plurality of polycationic/nucleic acid nanoparticles of claim 24, wherein the particle size is selected from the group consisting of about 300 nm, about 400 nm, and about 500 nm.

26. The plurality of polycationic/nucleic acid nanoparticles of claims 22-25, wherein the plurality of polycationic/nucleic acid nanoparticles has a polydispersity index of about 0.15±0.05 for a z-average particle size of 300 nm, a polydispersity index of about 0.25±0.05 for a z-average particle size of 400 nm, and a polydispersity index of about 0.35±0.05 for a z-average particle size of 500 nm.

27. A method for preparing a viral vector, the method comprising contacting one or more cells with a polycation/polyanion complex nanoparticle prepared by the method of any one of claims 1-21 or the plurality of polycationic/nucleic acid nanoparticles of any one of claims 22-26.

28. The method of claim 27, comprising dosing the plurality of polycation/polyanion complex nanoparticles to a monolayer culture of the one or more cells or a suspension culture of the one or more cells.

29. The method of claims 27-28, wherein the one or more cells comprise HEK293 cells or a derivative thereof.

30. The method of claim 27, wherein the one or more cells comprise HEK293T cells.

31. The method of claim 27, wherein the one or more cells comprise HEK293T cells adapted for suspension culture.
